(19) United States
(12) Patent Application Publication
(10) Pub. No.: US 2012/0149723 A1
(43) Pub. Date: Jun. 14, 2012

(54) 5-METHYL-PIPERIDINE DERIVATIVES AS OREXIN RECEPTOR ANTAGONISTS FOR THE TREATMENT OF SLEEP DISORDER

(76) Inventor: Romano Di Fabio, Verona (IT)

(21) Appl. No.: 13/391,694

(22) PCT Filed: Aug. 16, 2010
(86) PCT No.: PCT/EP2010/061879

§ 371 Date: Feb. 22, 2012

Related U.S. Application Data
(60) Provisional application No. 61/236,312, filed on Aug. 24, 2009.

(51) Int. Cl. A61K 31/506; A61K 31/4545; A61P 25/24; A61P 25/20; A61P 25/22; A61P 25/30; A61P 3/00; C07D 401/14; A61P 25/00

(52) U.S. Cl. 514/274; 544/333; 514/256; 544/296; 546/193; 514/318

(57) ABSTRACT

This invention relates to heteroaryloxy 5-methyl substituted piperidine derivatives and their use as pharmaceuticals.

This invention relates to heteroaryloxy 5-methyl substituted piperidine derivatives and their use as pharmaceuticals.

Many medically significant biological processes are mediated by proteins participating in signal transduction pathways that involve G-proteins and/or second messengers.

Polypeptides and polynucleotides encoding the human 7-transmembrane G-protein coupled neuropeptide receptor, orexin-1 (HFGAN72), have been identified and are disclosed in EP875565, EP875566 and WO 96/34877. Polypeptides and polynucleotides encoding a second human orexin receptor, orexin-2 (HFGANP), have been identified and are disclosed in EP893498.

Polypeptides and polynucleotides encoding polypeptides which are ligands for the orexin-1 receptor, e.g. orexin-A (Lig72A) are disclosed in EP849361.

The orexin ligand and receptor system has been well characterised since its discovery (see for example Sakurai, T. et al (1998) Cell, 92 pp 573 to 585; Smart et al (1999) British Journal of Pharmacology 128 pp 1 to 3; Willie et al (2001) Ann. Rev. Neurosciences 24 pp 429 to 458; Sakurai (2007) Nature Reviews Neuroscience 8 pp 171 to 181; Ohno and Sakurai (2008) Front. Neuroendocrinology 29 pp 70 to 87). From these studies it has become clear that orexins and orexin receptors play a number of important physiological roles in mammals and open up the possibility of the development of new therapeutic treatments for a variety of diseases and disorders as described hereinbelow.

Experiments have shown that central administration of the ligand orexin-A stimulated food intake in freely-feeding rats during a 4 hour time period. This increase was approximately four-fold over control rats receiving vehicle. These data suggest that orexin-A may be an endogenous regulator of appetite (Sakurai, T. et al (1998) Cell, 92 pp 573 to 585; Peyron et al (1998) J. Neurosciences 18 pp 9996 to 10015; Willie et al (2001) Ann. Rev. Neurosciences 24 pp 429 to 458). Therefore, antagonists of the orexin-A receptor(s) may be useful in the treatment of obesity and diabetes. In support of this it has been shown that orexin receptor antagonist SB334867 potently reduced hedonic eating in rats (White et al (2005) Peptides 26 pp 2231 to 2238) and also attenuated high-fat pellet self-administration in rats (Nair et al (2008) British Journal of Pharmacology, published online 28 Jan. 2008). The search for new therapies to treat obesity and other eating disorders is an important challenge. According to WHO definitions a mean of 35% of subjects in 39 studies were overweight and a further 22% clinically obese in westernised societies. It has been estimated that 5.7% of all healthcare costs in the USA are a consequence of obesity. About 85% of Type 2 diabetics are obese. Diet and exercise are of value in all diabetics. The incidence of diagnosed diabetes in westernised countries is typically 5% and there are estimated to be an equal number undiagnosed. The incidence of both diseases is rising, demonstrating the inadequacy of current treatments which may be either ineffective or have toxicity risks including cardiovascular effects. Treatment of diabetes with sulfonylureas or insulin can cause hypoglycaemia, whilst metformin causes GI side-effects. No drug treatment for Type 2 diabetes has been shown to reduce the long-term complications of the disease. Insulin sensitisers will be useful for many diabetics, however they do not have an anti-obesity effect.

As well as having a role in food intake, the orexin system is also involved in sleep and wakefulness. Rat sleep/EEG studies have shown that central administration of orexin-A, an agonist of the orexin receptors, causes a dose-related increase in arousal, largely at the expense of a reduction in paradoxical sleep and slow wave sleep 2, when administered at the onset of the normal sleep period (Hagan et al (1999) Proc. Natl. Acad. Sci. 96 pp 10911 to 10916). The role of the orexin system in sleep and wakefulness is now well established (Sakurai (2007) Nature Reviews Neuroscience 8 pp 171 to 181; Ohno and Sakurai (2008) Front. Neuroendocrinology 29 pp 70 to 87; Chemelli et al (1999) Cell 98 pp 437 to 451; Lee et al (2005) J. Neuroscience 25 pp 6716 to 6720; Piper et al (2000) European J Neuroscience 12 pp 726-730 and Smart and Jerman (2002) Pharmacology and Therapeutics 94 pp 51 to 61). Antagonists of the orexin receptors may therefore be useful in the treatment of sleep disorders including insomnia. Studies with orexin receptor antagonists, for example SB334867, in rats (see for example Smith et al (2003) Neuroscience Letters 341 pp 256 to 258) and more recently dogs and humans (Brisbare-Roch et al (2007) Nature Medicine 13(2) pp 150 to 155) further support this.

In addition, recent studies have suggested a role for orexin antagonists in the treatment of motivational disorders, such as disorders related to reward seeking behaviours for example drug addiction and substance abuse (Borgland et al (2006) Neuron 49(4) pp 589-601; Boutrel et al (2005) Proc. Natl. Acad. Sci. 102(52) pp 19168 to 19173; Harris et al (2005) Nature 437 pp 556 to 559).

International Patent Applications WO99/09024, WO99/58533, WO00/47577 and WO00/47580 disclose phenyl urea derivatives and WO00/47576 discloses quinolinyl cinnamide derivatives as orexin receptor antagonists. WO05/118548 discloses substituted 1,2,3,4-tetrahydroisoquinoline derivatives as orexin antagonists.

WO01/96302, WO02/44172, WO02/89800, WO03/002559, WO03/002561, WO03/032991, WO03/037847, WO03/041711 and WO08/038,251 all disclose cyclic amine derivatives.

WO04/026866 discloses dialkyl N-aroyl cyclic amines. We have now found that certain heteroaryloxy 5-methyl substituted piperidine derivatives have beneficial properties including, for example, increased potency compared to the prior art compounds. The compounds of the present invention have good bioavailability and brain penetration such properties make these heteroaryloxy 5-methyl substituted piperidine derivatives very attractive as potential pharmaceutical agents which may be useful in the prevention or treatment of obesity, including obesity observed in Type 2 (non-insulin-dependent) diabetes patients, sleep disorders, anxiety, depression, schizophrenia, drug dependency or compulsive behaviour. Additionally these compounds may be useful in the treatment of stroke, particularly ischemic or haemorrhagic stroke, and/or blocking the emetic response, i.e. useful in the treatment of nausea and vomiting.

Accordingly the present invention provides a compound of formula (I)

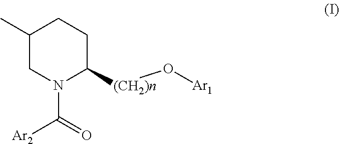

wherein:

Ar₂ is phenyl, pyridinyl, pyrimidinyl, pyridazinyl or pyrazinyl substituted with a group selected from C₁₋₄alkyl, halo, C₁₋₄alkoxy, haloC₁₋₄alkyl, haloC₁₋₄alkoxy and cyano, and is additionally substituted with a group Y where Y is phenyl, pyridinyl, pyrimidinyl, pyridazinyl, pyrazinyl, oxadiazolyl, phenyloxy, pyridinyloxy, pyrimidinyloxy, pyridazinyloxy, pyrazinyloxy, oxadiazolyloxy or a 5 membered heterocyclic group containing 1, 2, 3 or 4 heteroatoms selected from N, O or S, which group Y is optionally substituted with a group selected from C₁₋₄alkyl, haloC₁₋₄alkyl, C₁₋₄alkoxy, haloC₁₋₄alkoxy, cyano or halo;

Ar₁ is a heteroaryl group selected from the group consisting of pyridinyl, pyrimidinyl, pyridazinyl or pyrazinyl, which heteroaryl group is optionally substituted with 1, 2 or 3 substituents independently selected from the group consisting of: C₁₋₄alkyl, halo, C₁₋₄alkoxy, haloC₁₋₄alkyl, haloC₁₋₄alkoxy and cyano; or Ar1 is an 8 to 10 membered bicyclic heterocyclyl group which bicyclic heterocyclyl group is optionally substituted with C₁₋₄alkyl, haloC₁₋₄alkyl or halo;

n is 1 or 2;
or a pharmaceutically acceptable salt thereof.

In one embodiment Ar₂ is phenyl, pyridinyl, pyrimidinyl, pyridazinyl or pyrazinyl substituted with a group selected from C₁₋₄alkyl, halo, C₁₋₄alkoxy, haloC₁₋₄alkyl, haloC₁₋₄alkoxy and cyano, and is additionally substituted with a group Y where Y is phenyl, pyridinyl, pyrimidinyl, pyridazinyl, pyrazinyl, oxadiazolyl, phenyloxy, pyridinyloxy, pyrimidinyloxy, pyridazinyloxy, pyrazinyloxy, oxadiazolyloxy or a 5 membered heterocyclic group containing 1, 2, 3 or 4 heteroatoms selected from N, O or S, which group Y is optionally substituted with a group selected from C₁₋₄alkyl, haloC₁₋₄alkyl, C₁₋₄alkoxy, haloC₁₋₄alkoxy, cyano or halo; and Ar₁ is a heteroaryl group selected from the group consisting of pyridinyl, pyrimidinyl, pyridazinyl or pyrazinyl, which heteroaryl group is optionally substituted with 1, 2 or 3 substituents independently selected from the group consisting of: C₁₋₄alkyl, halo, C₁₋₄alkoxy, haloC₁₋₄alkyl, haloC₁₋₄alkoxy and cyano; and n is 1 or 2.

In one embodiment Ar₂ is pyridinyl substituted with a group selected from C₁₋₄alkyl, halo, C₁₋₄alkoxy, haloC₁₋₄alkyl, haloC₁₋₄alkoxy and cyano, and is additionally substituted with a group Y where Y is pyrimidinyl which is optionally substituted with a group selected from C₁₋₄alkyl, haloC₁₋₄alkyl, C₁₋₄alkoxy, haloC₁₋₄alkoxy, cyano or halo.

In another embodiment Ar₂ is pyridinyl substituted with C₁₋₄alkyl and is additionally substituted with a group Y where Y is pyrimidinyl.

In a further embodiment Ar₂ is pyridinyl substituted with methyl and is additionally substituted with a group Y where Y is pyrimidinyl.

In one embodiment Ar₁ is a heteroaryl group selected from the group consisting of pyridinyl, pyrimidinyl, pyridazinyl or pyrazinyl, which heteroaryl group is optionally substituted with 1, 2 or 3 substituents independently selected from the group consisting of: C₁₋₄alkyl, halo, C₁₋₄alkoxy, haloC₁₋₄alkyl, haloC₁₋₄alkoxy and cyano.

In another embodiment Ar₁ is pyridinyl which is optionally substituted with 1, 2 or 3 substituents independently selected from the group consisting of: C₁₋₄alkyl, halo, C₁₋₄alkoxy, haloC₁₋₄alkyl, haloC₁₋₄alkoxy and cyano.

In a further embodiment Ar₁ is pyridinyl which is substituted with 1 or 2 substituents independently selected from the group consisting of: C₁₋₄alkyl, halo and haloC₁₋₄alkyl.

In a still further embodiment Ar₁ is pyridinyl which is substituted with 1 or 2 substituents independently selected from the group consisting of: methyl, fluoro and trifluoromethyl.

In one embodiment Ar₂ is pyridinyl substituted with a group selected from C₁₋₄alkyl, halo, C₁₋₄alkoxy, haloC₁₋₄alkyl, haloC₁₋₄alkoxy and cyano, and is additionally substituted with a group Y where Y is pyrimidinyl which is optionally substituted with a group selected from C₁₋₄alkyl, haloC₁₋₄alkyl, C₁₋₄alkoxy, haloC₁₋₄alkoxy, cyano or halo; and Ar₁ is pyridinyl which is optionally substituted with 1, 2 or 3 substituents independently selected from the group consisting of: C₁₋₄alkyl, halo, C₁₋₄alkoxy, haloC₁₋₄alkyl, haloC₁₋₄alkoxy and cyano; and n is 1.

In another embodiment Ar₂ is pyridinyl substituted with C₁₋₄alkyl and is additionally substituted with a group Y where Y is pyrimidinyl; and Ar₁ is pyridinyl which is substituted with 1 or 2 substituents independently selected from the group consisting of: C₁₋₄alkyl, halo and haloC₁₋₄alkyl; and n is 1.

In a further embodiment Ar₂ is pyridinyl substituted with methyl and is additionally substituted with a group Y where Y is pyrimidinyl; and Ar₁ is pyridinyl which is substituted with 1 or 2 substituents independently selected from the group consisting of: methyl, fluoro and trifluoromethyl; and n is 1.

In one embodiment Ar₂ is pyridinyl substituted with a group selected from C₁₋₄alkyl, halo, C₁₋₄alkoxy, haloC₁₋₄alkyl, haloC₁₋₄alkoxy and cyano, and is additionally substituted with a group Y where Y is pyrimidinyl which is optionally substituted with a group selected from C₁₋₄alkyl, haloC₁₋₄alkyl, C₁₋₄alkoxy, haloC₁₋₄alkoxy, cyano or halo; and Ar₁ is pyridinyl which is optionally substituted with 1, 2 or 3 substituents independently selected from the group consisting of: C₁₋₄alkyl, halo, C₁₋₄alkoxy, haloC₁₋₄alkyl, haloC₁₋₄alkoxy and cyano; and n is 2.

In another embodiment Ar₂ is pyridinyl substituted with C₁₋₄alkyl and is additionally substituted with a group Y where Y is pyrimidinyl; and Ar₁ is pyridinyl which is substituted with 1 or 2 substituents independently selected from the group consisting of: C₁₋₄alkyl, halo and haloC₁₋₄alkyl; and n is 2.

In a further embodiment Ar₂ is pyridinyl substituted with methyl and is additionally substituted with a group Y where Y is pyrimidinyl; and Ar₁ is pyridinyl which is substituted with 1 or 2 substituents independently selected from the group consisting of: methyl, fluoro and trifluoromethyl; and n is 2.

In one embodiment the methyl at the 5 position on the piperidine ring is in the 5S configuration.

In one embodiment the invention provides the compound of formula (I) selected from the group consisting of:
2-(6-methyl-2-{[(2S,5S)-5-methyl-2-({[5-(trifluoromethyl)-2-pyridinyl]oxy}methyl)-1-piperidinyl]carbonyl}-3-pyridinyl)pyrimidine;
2-(6-methyl-2-{[(2S,5S)-5-methyl-2-({[4-(trifluoromethyl)-2-pyridinyl]oxy}methyl)-1-piperidinyl]carbonyl}-3-pyridinyl)pyrimidine;
2-(2-{[(2S,5S)-2-({[3-fluoro-5-(trifluoromethyl)-2-pyridinyl]oxy}methyl)-5-methyl-1-piperidinyl]carbonyl}-6-methyl-3-pyridinyl)pyrimidine;
2-{[((2 S,5S)-5-methyl-1-{[6-methyl-3-(2-pyrimidinyl)-2-pyridinyl]carbonyl}-2-piperidinyl)methyl]oxy}-5-(trifluoromethyl)pyrimidine;
2-{[((2S,5S)-5-methyl-1-{[6-methyl-3-(2-pyrimidinyl)-2-pyridinyl]carbonyl}-2-piperidinyl)methyl]oxy}-4-(trifluoromethyl)pyrimidine;
2-(2-{[(2S,5S)-2-({[2-chloro-4-(trifluoromethyl)-3-pyridinyl]oxy}methyl)-5-methyl-1-piperidinyl]carbonyl}-6-methyl-3-pyridinyl)pyrimidine;
2-{2-[((2S,5S)-2-{2-[(5-fluoro-2-pyridinyl)oxy]ethyl}-5-methyl-1-piperidinyl)carbonyl]-6-methyl-3-pyridinyl}pyrimidine; and
2-[((2S,5S)-2-{2-[(5-fluoro-2-pyridinyl)oxy]ethyl}-5-methyl-1-piperidinyl)carbonyl]-6-methyl-3-(2H-1,2,3-triazol-2-yl)pyridine;
   or a pharmaceutically acceptable salt thereof.

The Ar₁ group may be attached to the alkyloxy linker by means of a bond between the oxygen atom in said linker and any carbon or nitrogen atom in said Ar₁ ring. Preferably the Ar₁ group is attached to the linker by means of a bond between the oxygen atom in the linker and a carbon atom in the Ar₁ group ring.

Examples of a 5 membered heterocyclyl group containing 1, 2, 3 or 4 atoms selected from N, O or S include furanyl, thienyl, pyrrolyl, oxazolyl, thiazolyl, imidazolyl, oxadiazolyl, thiadiazolyl, triazolyl, triazinyl, tetrazolyl, isothiazolyl, isoxazolyl or pyrazolyl.

When the compound contains a C₁₋₄alkyl group, whether alone or forming part of a larger group, e.g. C₁₋₄alkoxy, the alkyl group may be straight chain, branched or cyclic, or combinations thereof. Examples of C₁₋₄alkyl are methyl or ethyl. An example of C₁₋₄alkoxy is methoxy.

Examples of haloC₁₋₄alkyl include trifluoromethyl (i.e. —CF₃).

Examples of C₁₋₄alkoxy include methoxy and ethoxy.

Examples of haloC₁₋₄alkoxy include trifluoromethoxy (i.e. —OCF₃).

Halogen or “halo” (when used, for example, in haloC₁₋₄)alkyl means fluoro, chloro, bromo or iodo.

It is to be understood that the present invention covers all combinations of particularised groups and substituents described herein above.

It will be appreciated that for use in medicine the salts of the compounds of formula (I) should be pharmaceutically acceptable. Suitable pharmaceutically acceptable salts will be apparent to those skilled in the art. Pharmaceutically acceptable salts include those described by Berge, Bighley and Monkhouse J. Pharm. Sci (1977) 66, pp 1-19. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and are included within the scope of this invention.

Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form and, if crystalline, may optionally be solvated, eg. as the hydrate. This invention includes within its scope stoichiometric solvates (eg. hydrates) as well as compounds containing variable amounts of solvent (eg. water).

It will be understood that the invention includes pharmaceutically acceptable derivatives of compounds of formula (I) and that these are included within the scope of the invention.

As used herein “pharmaceutically acceptable derivative” includes any pharmaceutically acceptable ester or salt of such ester of a compound of formula (I) which, upon administration to the recipient is capable of providing (directly or indirectly) a compound of formula (I) or an active metabolite or residue thereof.

The compounds of formula (I) are 2S enantiomers. Where additional chiral centres are present in compounds of formula (I), the present invention includes within its scope all possible enantiomers and diastereoisomers, including mixtures thereof. The different isomeric forms may be separated or resolved one from the other by conventional methods, or any given isomer may be obtained by conventional synthetic methods or by stereospecific or asymmetric syntheses. The invention also extends to any tautomeric forms or mixtures thereof.

The subject invention also includes isotopically-labeled compounds which are identical to those recited in formula (I) but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, ie. ³H, and carbon-14, ie. ¹⁴C, isotopes are particularly preferred for their ease of preparation and detectability. ¹¹C and ¹⁸F isotopes are particularly useful in PET (positron emission tomography).

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

According to a further aspect of the present invention there is provided a process for the preparation of compounds of formula (I) and derivatives thereof. The following schemes detail some synthetic routes to compounds of the invention. In the following schemes reactive groups can be protected with protecting groups and deprotected according to well established techniques.

### Schemes

According to a further feature of the invention there is provided a process for the preparation of compounds of formula (I) or salts thereof. The following is an example of a synthetic scheme that may be used to synthesise the compounds of the invention.

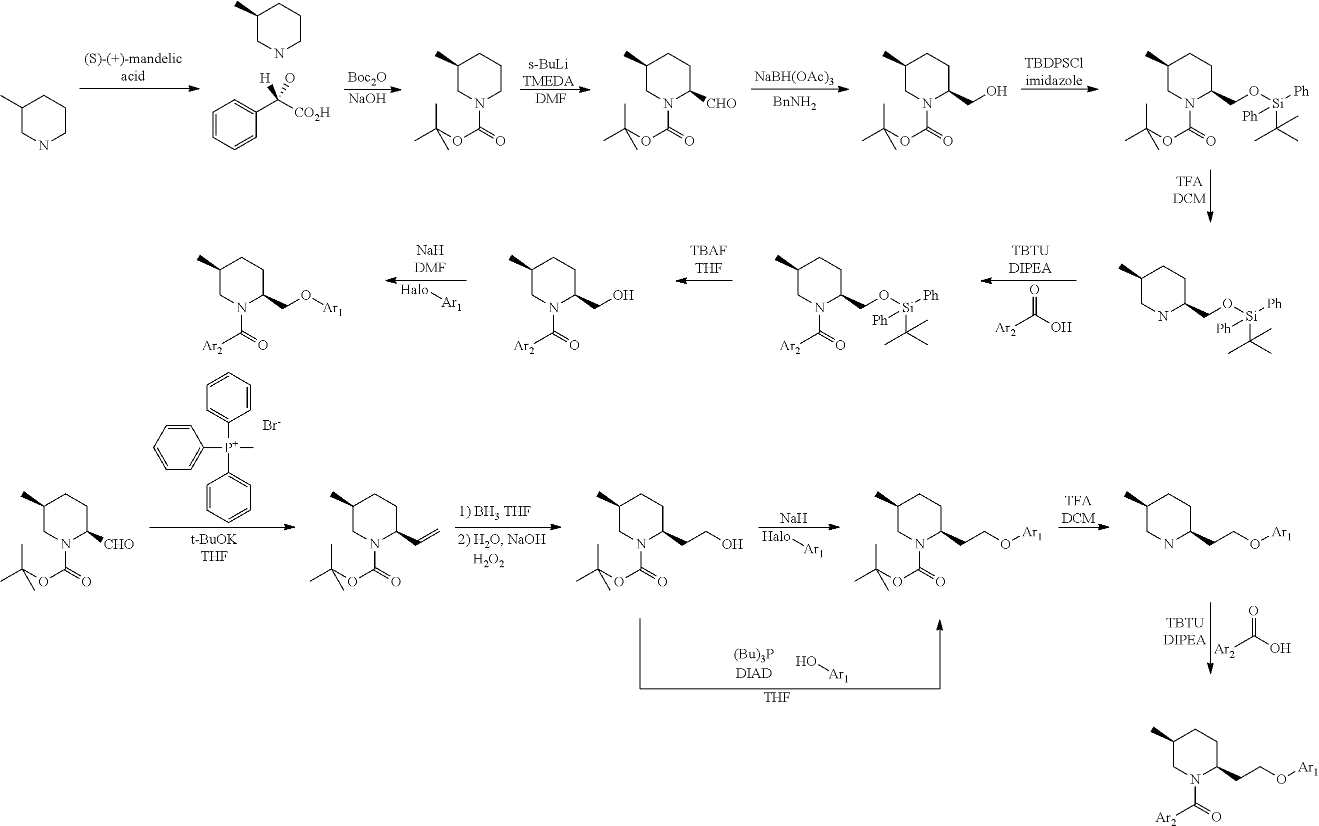

It will be understood by those skilled in the art that certain compounds of the invention can be converted into other compounds of the invention according to standard chemical methods.

The starting materials for use in the scheme are commercially available, known in the literature or can be prepared by known methods. For example (S)-(+)-mandelic acid [(S)-(α)-Hydroxyphenylacetic acid, Aldrich M2004] and 3 methyl piperidine (Aldrich M73001).

Pharmaceutically acceptable salts may be prepared conventionally by reaction with the appropriate acid or acid derivative.

The present invention provides compounds of formula (I) or a pharmaceutically acceptable salt thereof for use in human or veterinary medicine.

The compounds of formula (I) or their pharmaceutically acceptable salts may be of use for the treatment or prophylaxis of a disease or disorder where an antagonist of a human orexin receptor is required such as sleep disorders selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

In addition the compounds of formula (I) or their pharmaceutically acceptable salts may be of use for the treatment or prophylaxis of a disease or disorder where an antagonist of a human orexin receptor is required such as depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

Further, the compounds of formula (I) or their pharmaceutically acceptable salts may be of use for the treatment or prophylaxis of a disease or disorder where an antagonist of a human orexin receptor is required such as anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00).

In addition the compounds of formula (I) or their pharmaceutically acceptable salts may be of use for the treatment or prophylaxis of a disease or disorder where an antagonist of a human orexin receptor is required such as substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

In addition the compounds of formula (I) or their pharmaceutically acceptable salts may be of use for the treatment or prophylaxis of a disease or disorder where an antagonist of a human orexin receptor is required such as feeding disorders such as bulimia nervosa, binge eating, obesity, including obesity observed in Type 2 (non-insulin-dependent) diabetes patients. Further, the compounds of formula (I) or their pharmaceutically acceptable salts may be of use for the treatment or prophylaxis of a disease or disorder where an antagonist of a human orexin receptor is required such as stroke, particularly ischemic or haemorrhagic and/or in blocking an emetic response i.e. nausea and vomiting.

The numbers in brackets after the listed diseases refer to the classification code in DSM-IV: Diagnostic and Statistical Manual of Mental Disorders, 4th Edition, published by the American Psychiatric Association. The various subtypes of the disorders mentioned herein are contemplated as part of the present invention.

The invention also provides a method for the treatment of a disease or disorder where an antagonist of a human orexin receptor is required, for example those diseases and disorders mentioned hereinabove, in a subject in need thereof, comprising administering to said subject an effective amount of a compound of formula (I), or a pharmaceutically acceptable salt thereof.

The invention also provides a compound of formula (I), or a pharmaceutically acceptable salt thereof, for use in the treatment or prophylaxis of a disease or disorder where an antagonist of a human orexin receptor is required, for example those diseases and disorders mentioned hereinabove.

The invention also provides the use of a compound of formula (I), or a pharmaceutically acceptable salt thereof, in the manufacture of a medicament for use in the treatment or prophylaxis of a disease or disorder where an antagonist of a human Orexin receptor is required, for example those diseases and disorders mentioned hereinabove.

For use in therapy the compounds of the invention are usually administered as a pharmaceutical composition. The invention also provides a pharmaceutical composition comprising a compound of formula (I), or a pharmaceutically acceptable salt thereof, and a pharmaceutically acceptable carrier.

The compounds of formula (I) or their pharmaceutically acceptable salts may be administered by any convenient method, e.g. by oral, parenteral, buccal, sublingual, nasal, rectal or transdermal administration, and the pharmaceutical compositions adapted accordingly.

The compounds of formula (I) or their pharmaceutically acceptable salts which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

In one embodiment the composition is in unit dose form such as a tablet, capsule or ampoule.

The composition may contain from 0.1% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, of the active material, depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 500 mg, and such unit doses may be administered more than once a day, for example two or three a day. Such therapy may extend for a number of weeks or months.

Orexin-A (Sakurai, T. et al (1998) Cell, 92 pp 573-585) can be employed in screening procedures for compounds which inhibit the ligand's activation of the orexin-1 or orexin-2 receptors.

In general, such screening procedures involve providing appropriate cells which express the orexin-1 or orexin-2 receptor on their surface. Such cells include cells from mammals, yeast, Drosophila or E. coli. In particular, a polynucleotide encoding the orexin-1 or orexin-2 receptor is used to transfect cells to express the receptor. The expressed receptor is then contacted with a test compound and an orexin-1 or orexin-2 receptor ligand, as appropriate, to observe inhibition of a functional response. One such screening procedure involves the use of melanophores which are transfected to express the orexin-1 or orexin-2 receptor, as described in WO 92/01810.

Another screening procedure involves introducing RNA encoding the orexin-1 or orexin-2 receptor into Xenopus oocytes to transiently express the receptor. The receptor oocytes are then contacted with a receptor ligand and a test compound, followed by detection of inhibition of a signal in the case of screening for compounds which are thought to inhibit activation of the receptor by the ligand.

Another method involves screening for compounds which inhibit activation of the receptor by determining inhibition of binding of a labelled orexin-1 or orexin-2 receptor ligand to cells which have the orexin-1 or orexin-2 receptor (as appropriate) on their surface. This method involves transfecting a eukaryotic cell with DNA encoding the orexin-1 or orexin-2 receptor such that the cell expresses the receptor on its surface and contacting the cell or cell membrane preparation with a compound in the presence of a labelled form of an orexin-1 or orexin-2 receptor ligand. The ligand may contain a radioactive label. The amount of labelled ligand bound to the receptors is measured, e.g. by measuring radioactivity.

Yet another screening technique involves the use of FLIPR equipment for high throughput screening of test compounds that inhibit mobilisation of intracellular calcium ions, or other ions, by affecting the interaction of an orexin-1 or orexin-2 receptor ligand with the orexin-1 or orexin-2 receptor as appropriate.

Throughout the specification and claims which follow, unless the context requires otherwise, the word ‘comprise’, and variations such as ‘comprises’ and ‘comprising’ will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The following Examples illustrate the preparation of certain compounds of formula (I) or salts thereof. The Descriptions 1 to 23 illustrate the preparation of intermediates used to make compounds of formula (I) or salts thereof.

In the procedures that follow, after each starting material, reference to a description is typically provided. This is provided merely for assistance to the skilled chemist. The starting material may not necessarily have been prepared from the Description referred to.

The yields were calculated assuming that products were 100% pure if not stated otherwise.

The stereochemistry of the compounds of the Descriptions and Examples have been assigned on the assumption that the absolute configuration is maintained from the Description in which the chiral intermediate 1,1-dimethylethyl (2S,5S)-2-formyl-5-methyl-1-piperidinecarboxylate D3 is synthesized.

Compounds are named using ACD/Name PRO6.02 chemical naming software (Advanced Chemistry Development Inc., Toronto, Ontario, M5H2L3, Canada).

Proton Magnetic Resonance (NMR) spectra were recorded either on Varian instruments at 400, 500 or 600 MHz, or on a Bruker instrument at 400 MHz. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one is usually reported.

Unless otherwise specified, HPLC analyses indicated by HPLC (walk-up): rt (retention time)=x min, were performed on a Agilent 1100 series instrument using a Luna 3u C18(2) 100A column (50×2.0 mm, 3 μm particle size) [Mobile phase and Gradient: 100% (water+0.05% TFA) to 95% (acetonitrile+0.05% TFA) in 8 min. Column T=40° C. Flow rate=1 mL/min. UV detection wavelength=220 nm]. Other HPLC analyses, indicated by HPLC (walk-up, 3 min method), were performed using an Agilent Zorbax SB-C18 column (50×3.0 mm, 1.8 μm particle size) [Mobile phase and Gradient: 100% (water+0.05% TFA) to 95% (acetonitrile+0.05% TFA) in 2.5 min, hold 0.5 min. Column T=60° C. Flow rate=1.5 mL/min. UV detection wavelength=220 nm].

In the analytical characterization of the described compounds “MS” refers to Mass Spectra taken by Direct infusion Mass or to Mass Spectra associated with peaks taken by HPLC/MS or HPLC/MS analysis, where the Mass Spectrometer used is as mentioned below.

Direct infusion Mass spectra (MS) were run on a Agilent MSD 1100 Mass Spectrometer, operating in ES (+) and ES (−) ionization mode [ES (+): Mass range: 100-1000 amu. Infusion solvent: water+0.1% HCO₂H/CH₃CN 50/50. ES (−): Mass range: 100-1000 amu. Infusion solvent: water+0.05% NH₄OH/CH₃CN 50/50]

MS and UV spectra associated with the peaks were taken on an Agilent LC/MSD 1100 Mass Spectrometer coupled with HPLC instrument Agilent 1100 Series, operating in positive or negative electrospray ionization mode and in both acidic and basic gradient conditions [Acidic gradient LC/MS-ES (+ or −): analyses performed on a Supelcosil ABZ+Plus column (33×4.6 mm, 3 μm). Mobile phase: A-water+0.1% HCO₂H/B—CH₃CN. Gradient (standard method): t=0 min 0% (B), from 0% (B) to 95% (B) in 5 min lasting for 1.5 min, from 95% (B) to 0% (B) in 0.1 min, stop time 8.5 min. Column T=room temperature. Flow rate=1 mL/min. Gradient (fast method): t=0 min 0% (B), from 0% (B) to 95% (B) in 3 min lasting for 1 min, from 95% (B) to 0% (B) in 0.1 min, stop time 4.5 min. Column T=room temperature. Flow rate=2 mL/min.

Basic gradient LC/MS-ES (+ or −): analyses performed on a XTerra MS C18 column (30×4.6 mm, 2.5 μm). Mobile phase: A-5 mM aq. NH₄HCO₃+ammonia (pH 10)/B—CH₃CN. Gradient: t=0 min 0% (B), from 0% (B) to 50% (B) in 0.4 min, from 50% (B) to 95% (B) in 3.6 min lasting for 1 min, from 95% (B) to 0% (B) in 0.1 min, stop time 5.8 min. column temperature=room temperature. Flow rate=1.5 mL/min].

Mass range ES (+ or −): 100-1000 amu. UV detection range: 220-350 nm. The usage of this methodology is indicated by “LC-MS” in the analytic characterization of the described compounds.

Total ion current (TIC) and DAD UV chromatographic traces together with MS and UV spectra associated with the peaks were taken on a UPLC/MS Acquity™ system equipped with 2996 PDA detector and coupled to a Waters Micromass ZQ™ mass spectrometer operating in positive or negative electrospray ionisation mode [LC/MS-ES (+ or −): analyses performed using an Acquity™ UPLC BEH C18 column (50×21 mm, 1.7 μm particle size), column temperature 40° C.]. Mobile phase: A-water+0.1% HCOOH/B—CH₃CN+0.075% HCOOH, Flow rate: 1.0 mL/min, Gradient: t=0 min 3% B, t=0.05 min 6% B, t=0.57 min 70% B, t=1.4 min 99% B, t=1.45 min 3% B)]. The usage of this methodology is indicated by “UPLC” in the analytic characterization of the described compounds.

[LC/MS-ES (+ or −): analyses performed using an Acquity™ UPLC BEH C18 column (50×2.1 mm, 1.7 μm particle size) column temperature 40° C. Mobile phase: A-water+0.1% HCO₂H/B—CH₃CN+0.06% or 0.1% HCO₂H. Gradient: t=0 min 3% B, t=1.5 min 100% B, t=1.9 min 100% B, t=2 min 3% B stop time 2 min. Column T=40° C. Flow rate=1.0 mL/min. Mass range: ES (+): 100-1000 amu or ES(+): 50-800 amu. ES (−): 100-800 amu. UV detection range: 210-350 nm. The usage of this methodology is indicated by “UPLC (Acid IPQC)” in the analytic characterization of the described compounds.

[LC/MS-ES (+ or −): analyses performed using an Acquity™ UPLC BEH C18 column (50 x 2.1 mm, 1.7 μm particle size) column temperature 40° C. Mobile phase: A-water+0.1% HCO₂H/B—CH₃CN+0.06% or 0.1% HCO₂H. Gradient: t=0 min 3% B, t=0.05 min 6% B, t=0.57 min 70% B, t=1.06 min 99% B lasting for 0.389 min, t=1.45 min 3% B, stop time 1.5 min. Column T=40° C. Flow rate=1.0 mL/min. Mass range: ES (+): 100-1000 amu or ES(+): 50-800 amu, ES (−): 100-800 amu. UV detection range: 210-350 nm. The usage of this methodology is indicated by “UPLC (Acid QC_POS_{—}50-800 or GEN_QC or FINAL_QC)” in the analytic characterization of the described compounds.

[LC/MS-ES (+ or −): analyses performed using an Acquity™ UPLC BEH C18 column (50×2.1 mm, 1.7 μm particle size) column temperature 40° C. Mobile phase: A-water+0.1% HCO₂H/B—CH₃CN+0.06% or 0.1% HCO₂H. Gradient: t=0 min 3% B, t=1.06 min 99% B, t=1.45 min 99% B, t=1.46 min 3% B, stop time 1.5 min. Column T=40° C. Flow rate=1.0 mL/min. Mass range: ES (+): 100-1000 amu. ES (−): 100-800 amu. UV detection range: 210-350 nm. The usage of this methodology is indicated by “UPLC (Acid GEN_QC_SS)” in the analytic characterization of the described compounds.

Total ion current (TIC) and DAD UV chromatographic traces together with MS and UV spectra associated with the peaks were taken on a UPLC/MS Acquity™ system equipped with PDA detector and coupled to a Waters SQD mass spectrometer operating in positive and negative alternate electrospray ionisation mode [LC/MS-ES (+ or −): analyses performed using an Acquity™ UPLC BEH C18 column (50×2.1 mm, 1.7 μm particle size) column temperature 40° C. Mobile phase: A-10 mM aqueous solution of NH₄HCO₃ (adjusted to pH 10 with ammonia)/B—CH₃CN. Gradient: t=0 min 3% B, t=1.06 min 99% B lasting for 0.39 min, t=1.46 min 3% B, stop time 1.5 min. Column T=40° C. Flow rate=1.0 mL/min. Mass range: ES (+): 100-1000 amu or ES (+): 50-800 amu. ES (−): 100-1000 amu. UV detection range: 220-350 nm. The usage of this methodology is indicated by “UPLC (Basic GEN_QC or QC_POS_{—}50-800)” in the analytic characterization of the described compounds.

Unless otherwise specified, Preparative LC-MS purifications were run on a MDAP (Mass Detector Auto Purification) Waters instrument (MDAP FractionLynx). [LC/MS-ES (+): analyses performed using a Gemini C18 AXIA column (50×21 mm, 5 μm particle size). Mobile phase: A—NH₄HCO₃ sol. 10 mM, pH 10; B—CH₃CN. Flow rate: 17 mL/min. The gradient will be specified each time].

Preparative LC-MS purifications were also run on a MDAP (Mass Detector Auto Purification) Waters instrument. The usage of this methodology is indicated by “Fraction Lynx” in the analytic characterization of the described compounds.

For reactions involving microwave irradiation, a Personal Chemistry Emrys™ Optimizer was used.

In a number of preparations, purification was performed using Biotage manual flash chromatography (Flash+), Biotage automatic flash chromatography (Horizon, SP1 and SP4), Companion CombiFlash (ISCO) automatic flash chromatography, Flash Master Personal or Vac Master systems.

Flash chromatography was carried out on silica gel 230-400 mesh (supplied by Merck AG Darmstadt, Germany), Varian Mega Be—Si pre-packed cartridges, pre-packed Biotage silica cartridges (e.g. Biotage SNAP cartridge), KP-NH prepacked flash cartridges or ISCO RediSep Silica cartridges.

SPE-SCX cartridges are ion exchange solid phase extraction columns supplied by Varian. The eluent used with SPE-SCX cartridges is DCM and MeOH or only MeOH followed by 2 N ammonia solution in MeOH. The collected fractions are those eluted with the ammonia solution in MeOH.

SPE-Si cartridges are silica solid phase extraction columns supplied by Varian.

The following table lists the used abbreviations:
AcCl Acetyl chloride
ACN Acetonitrile
AcOH Acetic acid
atm atmosphere
bs or br.s broad signal
Boc t-Butoxycarbonyl
BnNH₂ Benzylamine
n-BuLi n-Butyl Lithium
s-BuLi s-Butyl Lithium
CV Column Volume
Cy Cyclohexanes
DCE 1,2-Dichloroethane
DCM Dichloromethane
DIAD Diterbutyl azadicarboxilate
DIPEA N,N-diisopropyl-N-ethylamine
DMF N,N-Dimethylformamide
DMSO Dimethylsulfoxide
Et₂O Diethylether
EtOAc Ethylacetate
eq. equivalent
MeOH Methanol
OAc Acetoxy
TBAF Tetrabutylamoniumfluoride
TBDPS tert-Butyl diphenylsilyl
TBTU O-(benzotriazol-1-yl)-N,N,N′N′-tetramethyluronium tetrafluoroborate
TEA Triethylamine
TFA Trifluoroacetic acid
THF Tetrahydrofuran
Wt Weight

### DESCRIPTIONS

### Description 1

### (2S)-hydroxy(phenyl)ethanoic acid-(3S)-3-methylpiperidine (1:1) (D1)

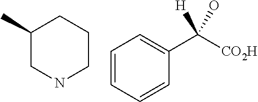

In a 10 L reactor, under nitrogen atmosphere, a solution of racemic 3-methylpiperidine (270 g, 2.72 mol) and (S)-(+)-mandelic acid (394 g, 2.59 mol) in MeOH (1 L) was cooled to 0° C. Without stirring, Et₂O (6.21 L) was added in several portions: (10×540 ml) every 20 minutes and 810 ml after 30 minutes from the last addition. After each addition of Et₂O, a short and slow stirring was applied in order to obtain a homogeneous phase. The final slurry was left standing overnight at 0° C. The precipitated solid was recovered by filtration, washed with cold Et₂O (2×540 ml) and dried under vacuum to afford the title compound D1 (150 g, 0.60 mol, 23% yield) [optical purity (94%) was determined by preparation of the Mosher amide derivative. The diastereomeric excess of the Mosher amide, determined via NMR spectroscopy, is representative of the enantiomeric excess of the precursor].

¹H-NMR (400 MHz, CDCl₃) δ ppm: 7.43-7.50 (m, 2H), 7.20-7.34 (m, 3H), 4.89 (s, 1 H), 2.89-3.05 (m, 2H), 2.17 (dt, 1H), 2.06 (t, 1H), 1.39-1.73 (m, 4H), 0.83-0.98 (m, 1H), 0.80 (d, 3H).

### Description 2

### 1,1-dimethylethyl (3S)-3-methyl-1-piperidinecarboxylate (D2)

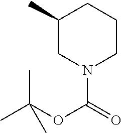

To a mixture of (2S)-hydroxy(phenyl)ethanoic acid-(3S)-3-methylpiperidine (1:1) D1 (150 g, 0.60 mol) in a 2.5 M NaOH aqueous solution (600 ml, 1.50 mol) cooled at 0° C., a solution of Boc₂O (130 g, 0.60 mol) in THF (1.2 L) was added dropwise over 1 hour (internal temperature kept below 9° C.) under vigorous stirring. Once the addition was completed, the mixture was allowed to reach room temperature and left under stirring overnight. Volatiles were evaporated and the aqueous phase extracted with Et₂O (3×500 ml). The collected organic phases were dried (Na₂SO₄), filtered and concentrated to dryness. The resulting crude material was eluted (Cy/EtOAc 90/10) through a silica gel pad to give the title compound D2 (103 g, 0.52 mol, 87% yield). MS: (ES/+) m/z: 200 (M+1). C₁₁H₂₁NO₂ requires 199.

¹H NMR (400 MHz, CDCl₃) δ ppm: 3.95 (bd, 2H), 2.70 (dt, 1H), 2.21-2.55 (m, 1H), 1.73-1.86 (m, 1H), 1.51-1.68 (m, 3H), 1.47 (s, 9H), 0.96-1.12 (m, 1H), 0.88 (d, 3H).

### Description 3

### 1,1-dimethylethyl (2S,5S)-2-formyl-5-methyl-1-piperidinecarboxylate (D3)

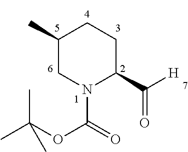

To a solution of 1,1-dimethylethyl (3S)-3-methyl-1-piperidinecarboxylate D2 (25 g, 0.13 mol) in anhydrous Et₂O (250 ml) cooled at −78° C. under nitrogen atmosphere, TMEDA (22.6 ml, 0.15 mol) was added followed by dropwise addition of s-BuLi (108 ml of a 1.4 M solution in Cy, 0.15 mol) over 40 min (exothermic addition: internal temperature kept below −70° C.). The pale yellow reaction mixture was left under stirring at −78° C. for 30 min then it was gradually warmed to −50° C. and stirred at this temperature for 30 min. The reaction was cooled again to −78° C., then TMEDA (further 0.3 eq) was added followed by dropwise addition of s-BuLi (further 0.3 eq.). The mixture was stirred for 30 min at −78° C., gradually warmed up to −50° C., stirred at this temperature for 30 min, then cooled to −78° C. Dry DMF (29.1 ml, 0.38 mol) was added dropwise (internal temperature kept below −70° C.). The resulting mixture was stirred for 30 minutes at −78° C. and then allowed to warm up to 0° C. The reaction mixture was quenched with a saturated NH₄Cl aqueous solution (200 ml) and water (100 ml). The layers were separated and the aqueous one back extracted with Et₂O (3×200 ml). The organic phases were collected, dried (Na₂SO₄), filtered and concentrated under vacuum to give a crude yellow oil. The material was purified by flash chromatography on silica gel (Biotage 75 L column, Cy/EtOAc 90/10). Collected fractions gave the title compound D3 (15 g, 0.066 mol, 53% yield).

¹H NMR [the relative stereochemistry of the compound was measured via NMR spectroscopy. The 1H spectrum shows that the compound gives rise to a mixture of two slowly exchanging conformers due to hindered rotation of the C═O group. 1H,1H scalar couplings [³J(H3,H2)˜5 Hz and ³J(H6ax,H5ax)˜12 Hz] and 1H,1H dipole correlation between H7 and H4ax determine that the six member ring bears a chair conformation with H2 in equatorial position and H5 in axial position. The relative stereochemistry is therefore SYN. The ANTI stereoisomer is present at ca. 25%. The ratio between the two diastereoisomers was determined on the ratio between integrals of proton signals H7 of each diastereoisomer. The absolute configuration is 2S, 5S on the assumption that the absolute configuration of D2 is retained. The assignment refers to the SYN isomer] (400 MHz, DMSO-d₆) δ ppm: 9.53 (d, 1H), 4.53-4.72 (m, 1H), 3.73-3.91 (m, 1H), 2.39 (t, 1H), 2.16-2.27 (m, 1H), 1.52-1.72 (m, 3H), 1.40 (s, 9H), 0.80 (d, 3H), 0.68-0.77 (m, 1H).

### Description 4

### 1,1-dimethylethyl (2S,5S)-2-(hydroxymethyl)-5-methyl-1-piperidinecarboxylate (D4)

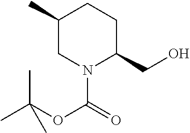

1,1-dimethylethyl (2S,5S)-2-formyl-5-methyl-1-piperidinecarboxylate D3 (2 g, 8.80 mmol), N10637-82-1 (0.79 g) and N2452-71-4 (1.21 g), was dissolved in MeOH (80 ml), the solution was chilled at 0° C. (ice bath) and sodium borohydride (0.399 g, 10.56 mmol) was added, after the addition the ice bath was removed. The reaction mixture was stirred at 23° C. for 30 min and then water (80 ml) was added. The organic solvent was removed under reduced pressure and the residual water was extracted with EtOAc, the organic layers were separated, dried (Na₂SO₄), filtered and evapoated giving a colorless oil the title compound D4 (2 g, 8.72 mmol, y=99%). MS: (ES/+) m/z: 534 (M+1). C₁₂H₂₃NO₃ requires 229.

### Description 5

### 1,1-dimethylethyl (2S,5S)-2-({[(1,1-dimethylethyl)(diphenyl)silyl]oxy}methyl)-5-methyl-1-piperidinecarboxylate (D5)

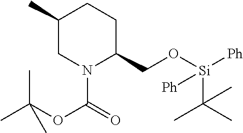

1,1-dimethylethyl (2S,5S)-2-(hydroxymethyl)-5-methyl-1-piperidinecarboxylate D4 (2 g, 8.72 mmol) was dissolved in DMF (55 ml) then imidazole (2.078 g, 30.5 mmol) and TBDPSC1 (2.464 ml, 9.59 mmol) were added and the reaction was stirred at room temperature for 3 hours. 200 ml of water were added and the product was extracted with Et₂O. All the organic fractions were collected together, dried over Na₂SO₄ anhydrous, filtered trough a phase separator tube and concentrated under vacuum to give a crude which was purified by silica gel chromatography (SNAP KP-Silica 100 g; eluted with Cy/EtOAc 15 CV from 100% Cy to 95/5) to afford the title compound D5 (3.5 g, 7.48 mmol, 86% yield). ¹H NMR (400 MHz, DMSO-d₆) δ ppm 7.54-7.72 (m, 4H), 7.36-7.53 (m, 6H), 4.11-4.37 (m, 1H), 3.55-3.89 (m, 3H), 2.11-2.30 (m, 1H), 1.13-1.98 (m, 14H), 1.00 (s, 9H), 0.69-0.86 (m, 3H).

### Description 6

### (2S,5S)-2-({[(1,1-dimethylethyl)(diphenyl)silyl]oxy}methyl)-5-methylpiperidine (D6)

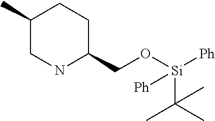

To an ice cooled solution of 1,1-dimethylethyl (2S,5S)-2-({[(1,1-dimethylethyl)(diphenyl)silyl]oxy}methyl)-5-methyl-1-piperidinecarboxylate D5 (3.5 g, 7.48 mmol) in DCM (75 ml) was added TFA (18.75 ml, 243 mmol). The ice bath was removed and the reaction mixture was stirred at 23° C. for 1.5 hours. After this time the volatiles were removed under reduced pressure to give a brown oil which was charged on a 20 g SCX, a yellow oil the title compound D6 (2.52 g, 6.86 mmol, 92% yield) was obtained. UPLC (Basic GEN_QC): rt=1.28 min, peak observed: 368 (M+1). C₂₃H₃₃NOSi requires 367. 1H NMR (400 MHz, DMSO-d6) δ ppm 0.93 (d, 3H) 1.00 (s, 9H) 1.26-1.75 (m, 5H) 1.93 (br. s., 1H) 2.52-2.59 (m, 1H) 2.60-2.71 (m, 2H) 3.43-3.61 (m, 2H) 7.36-7.53 (m, 6H) 7.55-7.71 (m, 4H)

### Description 7

### 3-(5,5-dimethyl-1,3,2-dioxaborinan-2-yl)-6-methyl-2-pyridinecarbonitrile (D7)

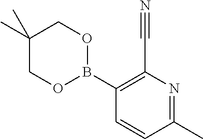

2,2,6,6-tetramethylpiperidine (3.49 ml, 20.52 mmol) was dissolved in dry THF (25 ml) under argon and stirred at −30° C.; BuLi (13.33 ml, 21.33 mmol) 1.6 M in hexane was added over 5 min (the temperature never exceeded −25° C.). The yellow solution was stirred at −30° C. for 20 min, then chilled at −78° C. and tris(1-methylethyl)borate (4.38 ml, 18.96 mmol) was added over 5 min (the temperature never exceeded −73° C.).

After 10 min at −78° C., 6-methyl-2-pyridinecarbonitrile (2.0 g, 16.93 mmol) dissolved in dry THF (14 ml) was added dropwise (over 20 min) maintaining internal temperature below −73° C. and the mixture became dark-brown. The mixture was stirred at −73° C. for 2 hours. The mixture was quenched with AcOH (2.374 ml, 41.5 mmol) dropwise at −73° C. (the temperature never exceeded −60° C. and the mixture became brilliant orange). The cooling bath was removed and the mixture left to reach the room temperature: during this period the mixture became thick and new THF (8 ml) had to be added in order to have a better stirring. The mixture was stirred 10 min at room temperature then 2,2-dimethyl-1,3-propanediol (2.409 g, 23.13 mmol) was added in one portion and the mixture stirred at room temperature overnight.

The solvent was evaporated and the orange residue taken-up with DCM (100 ml) and 10% water solution of KH₂PO₄ (100 ml). The phases were separated and the water phase was back-extracted with DCM (50 ml). The combined organic phases were washed with 10% water solution of KH₂PO₄ (50 ml). The DCM was evaporated. The residue was dissolved in Et₂O (100 ml) and extracted with NaOH 0.05 M (5×50 ml, boronic ester in water phase). The aqueous phases were joined together and the pH was adjusted between pH=4 and pH=5 with 10% water solution of KH₂PO₄ (50 ml). The so obtained yellow solution was extracted with AcOEt. All the organics joined together were dried (Na₂SO₄) and evaporated the title compound D7 2.29 g as yellow oil, that solidified on standing. C₁₂H₁₅BN₂O₂ requires 230. ¹HNMR (400 MHz, CDCl₃) δ ppm 7.97-8.15 (m, 1H), 7.31-7.36 (m, 1H), 3.85 (m, 4H), 2.52-2.73 (s, 3H), 0.97-1.10 (m, 6H).

### Description 8

### 6-methyl-3-(2-pyrimidinyl)-2-pyridinecarbonitrile (D8)

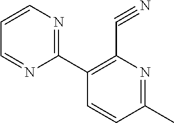

A) Isopropylmagnesium chloride-LiCl (37.9 ml, 36.5 mmol) was added portion wise (in overall 10 min) to a solution of 3-bromo-6-methyl-2-pyridinecarbonitrile (4 g, 20.30 mmol) in THF (150 ml) cooled to −70° C. (internal temperature). The reaction was kept to that temperature for 15 min. Then it was allowed to gently warm up to −40° C. in overall 1 hour. Then, it was cooled to −78° C. and zinc chloride (3.32 g, 24.36 mmol) was added. The resulting mixture was allowed to warm up to room temperature in 1 hour. Pd(Ph₃P)₄ (2.346 g, 2.030 mmol), 2-chloropyrimidine (3 g, 26.2 mmol) were added and the mixture was refluxed (external temperature 100° C.) until complete consumption of starting chloropyrimidine (3 hours). The reaction mixture was cooled to room temperature and poured into water (200 ml) cooled to 10° C. It was then extracted with EtOAc. The collected organic phases, containing large amount of colloid material and water, were washed with brine (200 ml). The water phase was filtered over a gouch, and the solid material was washed with further EtOAc. The collected organic phases were dried overnight over Na₂SO₄, filtered and concentrated to give (7 g) the crude material which was purified (Biotage Sp1 over a 240 g Silica Anolgix column, with a 25 g pre-column) to give the title compound D8 as yellow solid (1.8 g). UPLC (Acid GEN_QC_SS): rt=0.58 minutes, peak observed: 197 (M+1). C₁₁H₈N₄ requires 196.
B) 3-(5,5-dimethyl-1,3,2-dioxaborinan-2-yl)-6-methyl-2-pyridinecarbonitrile D7 (50.6 mg, 0.220 mmol) was dissolved 1,4-Dioxane (1 ml) under nitrogen in a vial, then 2-bromopyrimidine (42.0 mg, 0.264 mmol), CsF (67 mg, 0.441 mmol), Pd(Ph₃P)₄ (12 mg, 10.38 μmol) and CuI (7 mg, 0.037 mmol) were added in sequence. The vial was then capped and stirred at 65° C., after 1 hour the solvent was removed at reduced pressure and the residue partitioned between AcOEt and NaHCO₃ (saturated solution, 10 ml). The phases were separated and the water was extracted with AcOEt. The organic fraction were joined together, dried over Na₂SO₄ and evaporated at reduced pressure, obtaining an orange oily residue which was purified (Biotage, Snap 25 g silica gel column, from Cy to AcOEt/Cy 50:50) to obtain the title compound D8 as pail yellow solid (27.6 mg).

### Description 9

### 6-methyl-3-(2-pyrimidinyl)-2-pyridinecarboxylic acid (D9)

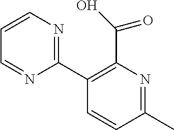

A) 6-methyl-3-(2-pyrimidinyl)-2-pyridinecarbonitrile D8 (0.8 g, 4.08 mmol) was reacted in 6 M aqueous HCl (40 ml, 240 mmol) at 80° C. for 3 hours, then solvent was removed under vacuum, and the resulting crude was purified (70 g Varian C18 column conditioning with MeOH, then water, loading in water, washing with water, product eluted with 100% MeOH) to give the title compound D9 (0.6 g) N11358-34-1 as yellow solid. UPLC (Acid GEN_QC_SS): rt=0.30 minutes, peak observed: 216 (M+1). C₁₁H₉N₃O₂ requires 217. ¹H NMR (400 MHz, DMSO-d₆) δ ppm 13.07 (bs, 1H), 8.78-9.01 (m, 2H), 8.39 (m, 1H), 7.39-7.67 (m, 2H), 2.56-2.67 (s, 3H).
B) 6-methyl-3-(2-pyrimidinyl)-2-pyridinecarbonitrile D8 (0.481 g, 2.451 mmol) was suspended in EtOH (5 ml) and a solution of NaOH (0.490 g, 12.26 mmol) in water (5 ml) was added. The yellow mixture was stirred at 100° C. overnight. The yellow solution was cooled to 25° C. and HCl 6 M (1.0 ml) was added dropwise till pH=4.5. The solvent was removed to give the title compound D9 a yellow powder that was dried at 50° C./vacuum for 1.5 hours to give 1.242 g.

### Description 10

### 2-(2-{[(2S,5S)-2-({[(1,1-dimethylethyl)(diphenyl)silyl]oxy}methyl)-5-methyl-1-piperidinyl]carbonyl}-6-methyl-3-pyridinyl)pyrimidine (D10)

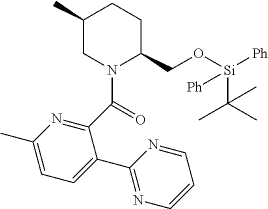

In a 500 ml round bottom flask, (2S,5S)-2-({[(1,1-dimethylethyl)(diphenyl)silyl]oxy}methyl)-5-methylpiperidine D6 (2.52 g, 6.86 mmol), 6-methyl-3-(2-pyrimidinyl)-2-pyridinecarboxylic acid D9 (4.61 g, 6.86 mmol) were dissolved in DCM (150 ml). To this solution DIPEA (3.59 ml, 20.57 mmol) and TBTU (2.421 g, 7.54 mmol) were added and the resulting mixture was stirred at room temperature. After 1 hour aqueous NaHCO₃ aqueous saturated solution was added and the organic layer was separated. The aqueous layer was backextracted with DCM and after the separation through a phase separator cartridge, the collected organic layers were evaporated under reduced pressure. The orange oil obtained was purified by silica gel chromatography (SNAP KP-NH, 50 g+100 g cartridges; eluted with Cy/EtOAc 1 CV 100% Cy, 3 CV from 100% to 80/20, 3 CV 80/20, 3 CV from 80/20 to 60/40, 5 CV 60/40) to give a yellowish the title compound D10 N10637-92-1 (2.5 g, 4.43 mmol, 64.6% yield) UPLC (Acid IPQC): rt=1.60 min, peak observed: 565 (M+1). C₃₄H₄₀N₄O₂Si requires 564. NMR

### Description 11

### ((2S,5S)-5-methyl-1-{[6-methyl-3-(2-pyrimidinyl)-2-pyridinyl]carbonyl}-2-piperidinyl)methanol (D11)

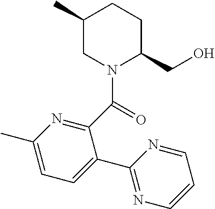

To a solution of 2-(2-{[(2S,5S)-2-({[(1,1-dimethylethyl)(diphenyl)silyl]oxy}methyl)-5-methyl-1-piperidinyl]carbonyl}-6-methyl-3-pyridinyl)pyrimidine D10 (2.5 g, 4.43 mmol) in THF (20 ml) at 20° C. TBAF 1 M in THF (4.43 ml, 4.43 mmol) was added. After 16 hours NH₄Cl (aqueous saturated solution 40 ml) was added and the mixture extracted with DCM, solvent was removed under reduced pressure and the residue was purified by silica gel chromatography (SNAP KP-Sil 50 g+100 g; eluted with 7 CV Cy/EtOAc 50/50, then with DCM/MeOH 3 CV 100% DCM, 5 CV 75/25, 10 CV 50/50) to give a yellowish solid the title compound D11 N10637-94-1 (1.3 g, 3.98 mmol, 90% yield) which was left at 40° C. under vacuum for 5 hours. UPLC (Acid IPQC): rt1=0.69 minutes and rt2=0.73 minutes (rotamers present), peaks observed: 327 (M+1). C₁₈H₂₂N₄O₂ requires 326. ¹H NMR (400 MHz, DMSO-d₆) δ ppm 8.88 (d, 2H), 8.44 (d, 1H), 7.40-7.50 (m, 2H), 4.63 (t, 1H), 4.30-4.39 (m, 1H), 3.47-3.73 (m, 3H), 2.54 (s, 3H), 2.38 (t, 1H), 1.22-1.75 (m, 5H), 0.93 (d, 3H).

### Description 12

### 1,1-dimethylethyl (2S,5S)-2-ethenyl-5-methyl-1-piperidinecarboxylate (D12)

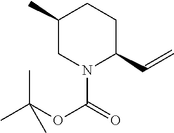

To a suspention of methyltriphenylphosphonium bromide (22.00 g, 61.6 mmol) in THF (200 ml) at room temperature, KOtBu (6.91 g, 61.6 mmol) was added (yellow suspention). The mixture was heated to 50° C. for 2 hours. Then at room temperature 1,1-dimethylethyl (2S,5S)-2-formyl-5-methyl-1-piperidinecarboxylate D3 (7 g, 30.8 mmol) was added and the reaction mixture stirred for 2 hours. NaHCO₃ aqueous saturated solution was added and the mixture extracted twice with DCM. The collected DCM phase was dried and the solvent removed to give the crude product which was purified by silica gel flash chromatography (the crude was divided in two portions and each portion purified by 340 g column gradient elution from Cy to Cy/EtOAc 70/30) to give the title compound D12 N10649-58-1 (4.23 g, 18.77 mmol, 61% yield) as yellow oil. UPLC (Acid GEN_QC): rt1=0.91 minutes, peaks observed: 226 (M+1). C₁₃H₂₃NO₂ requires 225. ¹H NMR (500 MHz, DMSO-d₆) δ ppm 5.67-5.90 (m, 1H), 5.13-5.19 (m, 1H), 4.92-5.00 (m, 1H), 4.54-4.74 (m, 1H), 3.61-3.90 (m, 1H), 2.22-2.46 (m, 1H), 1.38-1.85 (m, 4H), 1.38 (s, 9H), 0.97-1.10 (m, 1H), 0.80 (d, 3H).

### Description 13

### 1,1-dimethylethyl (2S,5S)-2-(2-hydroxyethyl)-5-methyl-1-piperidinecarboxylate (D13)

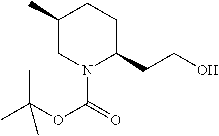

To a solution of 1,1-dimethylethyl (2S,5S)-2-ethenyl-5-methyl-1-piperidinecarboxylate D12 (2 g, 8.88 mmol) in THF (40 ml) stirred under nitrogen at room temperature was added a solution of BH₃. THF 1 M in THF (26.6 mL, 26.6 mmol) dropwise. The reaction mixture was stirred at 70° C. for 20 hours. Then at room temperature water (0.480 ml, 26.6 mmol) was added (caution!) and NaOH 1 M in water (3.55 mL, 3.55 mmol) and slowly H₂O₂ 30% Wt in water (1.360 ml, 13.31 mmol) the reaction mixture was stirred at room temperature for 2 hours. Et₂O was added and then water, the aqueous extracted with Et₂O, the phases were separated, the combined organic solvents were passed through a hydrophobic filter and the organic solvent removed to give the crude which was purified by flash chromatography (silica 100 g column, gradient elution from Cy to Cy/EtOAc 65/35) to afford the title compound D13 N10649-63-1 (1.25 g, 5.14 mmol, 58% yield) as yellow oil. MS: (ES/+) m/z: 244 (M+1). C₁₃H₂₅NO₃ requires 243. ¹H NMR (400 MHz, DMSO-d₆) δ ppm 4.27-4.46 (m, 1H), 3.66-3.96 (m, 2H), 3.34-3.49 (m, 2H), 2.21-2.45 (m, 1H), 1.65-1.95 (m, 2H), 1.46-1.65 (m, 3H), 1.16-1.45 (m, 11H), 0.76-0.95 (m, 3H).

### Description 14

### 1,1-dimethylethyl (2S,5S)-2-{2-[(5-fluoro-2-pyridinyl)oxy]ethyl}-5-methyl-1-piperidinecarboxylate (D14)

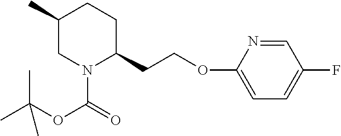

To a solution of 1,1-dimethylethyl (2S,5S)-2-(2-hydroxyethyl)-5-methyl-1-piperidinecarboxylate D13 (1 g, 4.11 mmol), 5-fluoro-2(1H)-pyridinone (0.465 g, 4.11 mmol) and tributylphosphane (2.028 ml, 8.22 mmol) in THF (30 ml) stirred under nitrogen at 50° C. was added a solution of DIAD (1.892 g, 8.22 mmol) in THF 10 ml. The reaction mixture was stirred at 50° C. for 18 hours. The volatiles were removed to give the crude which was purified by flash chromatography (silica 120 g column, gradient elution from Cy to Cy/EtOAc 80/20) to afford the title compound D14 N10649-64-1 (753 mg, 2.225 mmol, 54% yield). UPLC (Acid GEN_QC): rt1=0.97 minutes, peaks observed: 339 (M+1). C₁₈H₂₇FN₂O₃ requires 338. ¹H NMR (400 MHz, DMSO-d₆) δ ppm 8.05-8.16 (m, 1H), 7.61-7.74 (m, 1H), 6.77-6.86 (m, 1H), 4.25-4.42 (m, 1H), 4.05-4.24 (m, 2H), 3.66-3.97 (m, 1H), 2.28-2.40 (m, 1H), 2.01-2.25 (m, 1H), 1.67-1.87 (m, 1H), 1.28-1.60 (m, 5H), 1.24-1.29 (m, 9H), 0.84 (d, 3H).

### Description 15

### 5-fluoro-2-({2-[(2S,5S)-5-methyl-2-piperidinyl]ethyl}oxy)pyridine (D15)

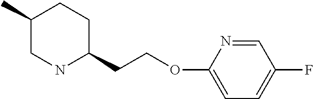

To a solution of 1,1-dimethylethyl (2S,5S)-2-{2-[(5-fluoro-2-pyridinyl)oxy]ethyl}-5-methyl-1-piperidinecarboxylate D14 (750 mg, 2.216 mmol), in DCM (30 ml) stirred at room temperature was added TFA (1.707 ml, 22.16 mmol) in DCM (30 ml). The reaction mixture was stirred at room temperature for 18 hours. Then the volatiles were removed to give the crude product which was purified by SCX 20 g the product so obtained was purified by silica flash chromatography (10 g column, EtOAc/Cy to EtOAc to EtOAc/MeOH 70/30) to afford the title compound D15 N10649-65-1 (520 mg, 2.182 mmol, 98% yield). UPLC (method: Acid GEN_QC): rt1=0.47 minutes, peaks observed: 239 (M+1). C₁₃H₁₉FN₂O requires 238.

### Description 16

### 2-methylfuro[3,4-b]pyridine-5,7-dione (D16)

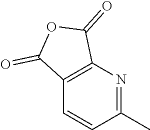

In a 100 ml round-bottomed flask 6-methyl-2,3-pyridinedicarboxylic acid (10 g, 55.2 mmol) and acetic anhydride (26 ml, 276 mmol) were added and heated at 100° C. under nitrogen for 5 hours. After this time the volatiles were removed under vacuum to give the title compound D16 (8.2 g, 50.3 mmol, 91% yield) as a slightly brown solid. ¹H NMR (400 MHz, DMSO-d₆) δ ppm 8.41 (d, 1H), 7.82 (d, 1H), 2.73 (s, 3H).

### Description 17

### 6-methyl-2-[(methyloxy)carbonyl]-3-pyridinecarboxylic acid (D17)

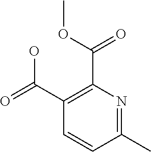

2-methylfuro[3,4-b]pyridine-5,7-dione D16 (3 g, 18.39 mmol) was added portionwise over 5 minutes to stirred MeOH (20 ml) at 0° C. The mixture was stirred at 0° C. for 30 minutes then at room temperature for other 2.5 hours. The solution was evaporated at reduced pressure and the residue recrystallized from toluene (50 ml). The solid was filtered and dried under high vacuum for 30 minutes, obtaining a first batch of the title compound D17 (1.16 g) as pale brown solid. From the toluene solution new solid precipitated: this solid was filtered and dried under high vacuum for 30 minutes, obtaining a second batch of the title compound D17 (352 mg) as pale yellow solid. The toluene solution was then evaporated at reduced pressure and the residue recrystallized again from toluene (25 ml). The solid was filtered and dried under high vacuum for 30 minutes, obtaining a third batch of the title compound D17 (615 mg) as pale yellow solid. UPLC (Basic GEN_QC): rt=0.23 minutes, peak observed: 195 (M+1). C₉H₉NO₄ requires 196. ¹H NMR (400 MHz, DMSO-d₆) δ ppm 13.61 (br. s., 1H), 8.09-8.31 (m, 1H), 7.51 (m, 1H), 3.82 (s, 3H), 2.55 (s, 3H).

### Description 18

### methyl 3-({[(1,1-dimethylethyl)oxy]carbonyl}amino)-6-methyl-2-pyridinecarboxylate (D18)

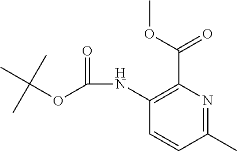

6-methyl-2-[(methyloxy)carbonyl]-3-pyridinecarboxylic acid D17 (1.15 g, 5.89 mmol) was suspended in toluene (40 ml) and DIPEA (1.25 ml, 7.16 mmol) was added, causing the complete dissolution of the solid. This mixture was stirred 10 minutes at room temperature, then diphenyl azidophosphate (1.35 ml, 6.26 mmol) was added in one portion and the mixture was stirred at reflux for 1 hour. The solution was cooled at room temperature and t-BuOH (2.5 ml, 26 mmol) was added in one portion.

The mixture was then stirred at 70° C. for 1 hour and then cooled at room temperature, Et₂O (50 ml) was added and the resulting solution washed with NaHCO₃ aqueous saturated solution. The aqueous phases were joined together and back-extracted with Et₂O. The two organic solutions were joined together, dried over Na₂SO₄ and evaporated at reduced pressure, obtaining the crude target material as pale yellow oil. This material was purified by flash chromatography on silica gel (Biotage, EtOAc/Cy from 10/90 to 70/30; Snap-100 g column). The title compound D18 was obtained (1.315 g) as white solid. UPLC (Basic GEN_QC): rt=0.68 minutes, peak observed: 267 (M+1). C₁₃H₁₈N₂O₄ requires 266. ¹H NMR (400 MHz, CDCl₃) δ ppm 10.13 (bs., 1H), 8.77 (d, 1H), 7.34 (d, 1H), 4.03 (s, 3H), 2.59 (s, 3H), 1.53-1.56 (m, 9H).

### Description 19

### methyl 3-amino-6-methyl-2-pyridinecarboxylate (D19)

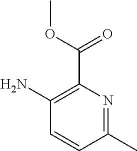

Methyl 3-({[(1,1-dimethylethyl)oxy]carbonyl} amino)-6-methyl-2-pyridinecarboxylate D18 (1.3 g, 4.88 mmol) was dissolved in DCM (80 ml) and the mixture stirred at 0° C. A solution of TFA (5 ml, 64.9 mmol) in DCM (10 ml) was dropped into the cold mixture over 3 minutes. The resulting solution was left under stirring at 0° C. for 30 minutes, then the mixture was left still at room temperature overnight. TFA (4 ml, 51.9 mmol) dissolved in DCM (10 ml) was added over 3 minutes and the mixture stirred again at room temperature for 5 hours. The solution was loaded onto an SCX-25 g column to afford the title compound D19 (770 mg, 4.63 mmol, 95% yield) was obtained as a white solid. UPLC (Basic GEN_QC): rt=0.44 minutes, peak observed: 167 (M+1). C₈H₁₀N₂O₂ requires 166. ¹H NMR (400 MHz, CDCl₃) δ ppm 7.14 (d, 1H), 7.01 (d, 1H), 3.99 (s, 3H), 2.52 (s, 3H).

### Description 20

### methyl 3-iodo-6-methyl-2-pyridinecarboxylate (D20)

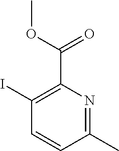

HCl 6 M solution in water (4.5 ml, 27.0 mmol) was added to methyl 3-amino-6-methyl-2-pyridinecarboxylate D19 (768 mg, 4.62 mmol) and the resulting pale yellow mixture was sequentially diluted with water (4×5 ml) and chilled at 0° C. (internal temperature). A solution of sodium nitrite (480 mg, 6.96 mmol) in water (2 ml) was dropped into the mixture over 1 minute. After this addition the mixture was stirred at 0° C. for 30 minutes, then a solution of KI (1.69 g, 10.18 mmol) in water (2 ml) was added over 1 minute, causing the formation of a dark violet crust (moderate gas evolution). The mixture was left under stirring for 1 hour: during this period the temperature passed from 0° C. to +5° C. EtOAc (50 ml) was then added to the stirred mixture, causing the dissolution of the dark solid. Water (50 ml) and EtOAc (50 ml) were added and the whole mixture was poured into a separator funnel. After the separation of the two phases, the water phase was extracted with EtOAc. All the organic phases were joined together and washed with NaHCO₃ saturated solution; the acidic water phase was neutralized by the addition of the previously used NaHCO₃ saturated solution and the resulting mixture extracted with EtOAc. All the organic phases were joined together, dried over Na₂SO₄ and evaporated at reduced pressure, obtaining the crude target material as dark brown/violet oil. This material was purified by silica gel chromatography (Biotage SP4 Snap-100 g column, EtOAc/Cy from 10/90 to 30/70). The title compound D₂O was obtained as a pale brown solid (1.1 g, 3.99 mmol, 86% yield). UPLC (Basic GEN_QC): rt=0.68 minutes, peak observed: 278 (M+1). C₈H₈INO₂ requires 277. ¹H NMR (400 MHz, CDCl₃) δ ppm 8.12 (d, 1H), 7.01 (d, 1H), 4.01 (s, 3H), 2.58 (s, 3H).

### Description 21

### methyl 6-methyl-3-(2H-1,2,3-triazol-2-yl)-2-pyridinecarboxylate (D21)

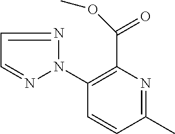

DMF (1.5 ml) was added to a mixture of methyl 3-iodo-6-methyl-2-pyridinecarboxylate D₂O (100 mg, 0.361 mmol), 1H-1,2,3-triazole (49.9 mg, 0.722 mmol), (1R,2R)—N,N′-dimethyl-1,2-cyclohexanediamine (10.27 mg, 0.072 mmol), CuI (3.44 mg, 0.018 mmol) and Cs₂CO₃ carbonate (235 mg, 0.722 mmol) in a microwave vial. The mixture was degassed via three vacuum/nitrogen cycles then irradiated in a single mode microwave reactor to 120° C. for 20 minutes. The mixture was irradiated in a single mode microwave reactor to 120° C. for a further 40 minutes. The reaction mixture was cooled and filtered washing the solids with EtOAc. The solids were dissolved in pH=3 buffer solution (5 ml); UPLC check of this aqueous solution showed it contained a considerable quantity of 6-methyl-3-(2H-1,2,3-triazol-2-yl)-2-pyridinecarboxylic acid. The aqueous phase was extracted repeatedly with DCM; the combined DCM extracts were diluted with MeOH (50 ml) and treated with TMS-diazomethane. The volatiles were evaporated to give a yellow residue that was purified by flash chromatography on silica gel (Biotage, SNAP 10 g column, 10%-50% EtOAc/cyclohexane) to give the title compound D21 (38 mg, 0.174 mmol, 48% yield) as a white solid. UPLC: rt=0.57 minutes. peak observed: 219 (M+1). C₁₀H₁₀N₄O₂ requires 218. ¹H NMR (400 MHz, CDCl₃) δ ppm 8.20 (d, 1H), 7.87 (s, 2H), 7.44 (d, 1H), 3.94 (s, 3H), 2.71 (s, 3H).

### Description 22

### 6-methyl-3-(2H-1,2,3-triazol-2-yl)-2-pyridinecarboxylic acid (D22)

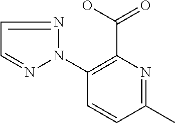

A solution of methyl 6-methyl-3-(2H-1,2,3-triazol-2-yl)-2-pyridinecarboxylate D21 (36 mg, 0.165 mmol) and LiOH (5.93 mg, 0.247 mmol) in THF/water (2:1, 3 ml) was stirred overnight. The mixture was evaporated under reduced pressure; the residue was taken up in water (2 ml) and neutralised with 1 M HCl water solution and then loaded onto a pre-conditioned C18 column (5 g). The column was eluted with water and then MeOH. The methanol fractions were evaporated under reduced pressure to give the title compound D22 (34 mg, 0.158 mmol, 95% purity by NMR, 96% yield) as a white solid. UPLC: rt=0.30 minutes. peak observed: 205 (M+1). C₉H₈N₄O₂ requires 204. ¹H NMR (400 MHz, MeOD) δ ppm 8.24 (d, 1H), 7.99 (s, 2H), 7.61 (d, 1H), 2.67 (s, 3H).

### Description 23

### 2-({[(2S,5S)-5-methyl-2-piperidinyl]methyl}oxy)-5-(trifluoromethyl)pyridine (D23)

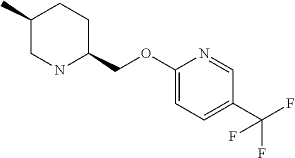

To a solution of 1,1-dimethylethyl (2S,5S)-2-(hydroxymethyl)-5-methyl-1-piperidinecarboxylate D4 (400 mg, 1.744 mmol) in THF (10 ml) stirred under nitrogen at room temperature was added NaH (46.0 mg, 1.919 mmol) in one charge, the reaction mixture was stirred at room temperature for 20 minutes and then 2-chloro-5-(trifluoromethyl)pyridine (380 mg, 2.093 mmol) was added and the reaction mixture was heated at 80° C. for 4 hours. Then aqueous saturated solution of NaHCO₃ was added and THF was removed under reduced pressure, the acqueous extracted with DCM. The combined organic solvents were removed to give the crude product which was redissolved in dry DCM and TFA (1.344 ml, 17.44 mmol) was added, the mixture was stirred at room temperature for 5 hours. Then the solvent was removed and cold NH₃ 2 M in MeOH was added the white precipitate was filtered and washed with DCM. The organic filtrate was evaporated to give the crude N10649-48-1 which was purified on silica (10 g column, EtOAC to EtOAc/MeOH 8:2) to afford the title compound D23 (80 mg, 0.292 mmol, 17% yield). UPLC (Basic GEN_QC): rt=0.99 min, peaks observed: 275 (M+1). C₁₃H₁₇F₃N₂O requires 274. ¹H NMR (400 MHz, CDCl₃) δ ppm 8.37 (s, 1H), 7.77 (m, 1H), 6.88 (m, 1 H), 4.44-4.69 (m, 2H), 3.57 (m, 1H), 3.11 (m, 1H), 2.88 (m, 1H), 2.01-2.13 (m, 1H), 1.82-2.01 (m, 2H), 1.63-1.82 (m, 1H), 1.44-1.63 (m, 1H), 1.01-1.13 (m, 3H).

### EXAMPLES

### Example 1

### 2-(6-methyl-2-{[(2S,5S)-5-methyl-2-({[5-(trifluoromethyl)-2-pyridinyl]oxy}methyl)-1-piperidinyl]carbonyl}-3-pyridinyl)pyrimidine (E1)

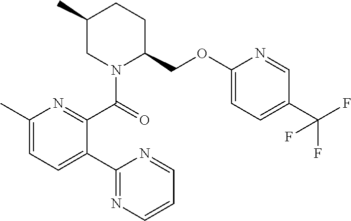

To a solution of 2-({[(2S,5S)-5-methyl-2-piperidinyl]methyl}oxy)-5-(trifluoromethyl)pyridine D23 (80 mg, 0.292 mmol), DIPEA (0.153 ml, 0.875 mmol) and 6-methyl-3-(2-pyrimidinyl)-2-pyridinecarboxylic acid D9 (209 mg, 0.292 mmol) in DCM (10 ml) stirred under nitrogen at room temperature was added TBTU (112 mg, 0.350 mmol) in one charge. The reaction mixture was stirred at room temperature for 18 hours. NaHCO₃ aqueous satured solution was added and the acqueous extracted with DCM, the phases were separated on a hydrophobic frit and the combined organic solvent was removed to give the crude product which was purified by flash chromatography (silica —NH 50 g column, gradient elution from Cy to Cy/EtOAc 4:6) to afford the product which was purified by flash chromatography (silica —NH 28 g column, gradient elution from Cy to Cy/EtOAc 4:6) to afford the title compound E1 (47 mg, 0.100 mmol, 34% yield). UPLC (Basic GEN_QC): rt1=0.94 minutes and rt2=0.97 min (rotamers present), peaks observed: 472 (M+1). C₂₄H₂₄F₃N₅O₂ requires 471. ¹H NMR (500 MHz, CDCl₃) δ ppm 8.85 (d, 2H), 8.61 (d, 1 H), 8.50 (s, 1H), 7.80 (dd, 1H), 7.17-7.32 (m, 2H), 6.90 (d, 1H), 5.19-5.26 (m, 1H), 4.51-4.78 (m, 2H), 3.25 (dd, 1H), 2.75 (t, 1H), 2.64 (s, 3H), 2.04-2.14 (m, 1H), 1.85-1.96 (m, 1H), 1.53-1.83 (m, 2H), 1.24-1.44 (m, 1H), 0.75 (d, 3H).

### Example 2

### 2-(6-methyl-2-{[(2S,5S)-5-methyl-2-({[4-(trifluoromethyl)-2-pyridinyl]oxy}methyl)-1-piperidinyl]carbonyl}-3-pyridinyl)pyrimidine (E2)

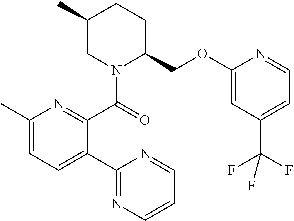

To a solution of ((2S,5S)-5-methyl-1-{[6-methyl-3-(2-pyrimidinyl)-2-pyridinyl]carbonyl}-2-piperidinyl)methanol D11 (50 mg, 0.153 mmol) in dry DMF (2 ml at 0° C. under N₂ flux, sodium hydride (6.74 mg, 0.169 mmol) was added and mixture was stirred at room temperature for 5 min, then 2-fluoro-4-(trifluoromethyl)pyridine (30.3 mg, 0.184 mmol) was added and the mixture was warmed to 55° C. and stirred at that temperature for 1 hour. After cooling the mixture was diluted with Et₂O and washed with water. The organic phase was dried and evaporated and the resulting crude purified by flash chromatography on silica (KP-Sil column SNAP 10 g eluting with DCM/MeOH 9:1) affording the title compound E2 (60 mg, 0.127 mmol, 83% yield) as white foam. UPLC (Acid GEN_QC_SS): rt1=0.99 minutes and rt2=1.0 min (rotamers present), peaks observed: 472 (M+1). C₂₄H₂₄F₃N₅O₂ requires 471.

¹H NMR (400 MHz, CDCl₃) δ ppm 0.76 (d, 3H) 1.03 (d, 2H) 1.24-1.49 (m, 2H) 1.64-1.83 (m, 5H) 1.83-2.01 (m, 1H) 2.10 (br. s., 1H) 2.50-2.62 (m, 3H) 2.62-2.69 (m, 3H) 2.70-2.82 (m, 1H) 3.25 (d, 1H) 4.01 (br. s., 1H) 4.51 (dd, 1H) 4.59-4.83 (m, 4H) 5.23 (br. s., 1H) 6.86 (s, 1H) 7.00-7.07 (m, 2H) 7.11 (d, 1H) 7.21 (q, 2H) 7.31 (d, 2H) 8.14 (d, 1H) 8.37 (d, 1H) 8.55 (d, 1H) 8.61 (d, 1H) 8.77 (d, 1H) 8.85 (d, 2H)

The following compounds were prepared using a similar procedure to that described for Example 2. Each compound was obtained by reacting ((2S,5S)-5-methyl-1-{[6-methyl-3-(2-pyrimidinyl)-2-pyridinyl]carbonyl}-2-piperidinyl)methanol D11 with the appropriate halo derivative. This is provided merely for assistance to the skilled chemist. The starting material may not necessarily have been prepared from the batch referred to.

| No. | Reactants | Characterising data |
|---|---|---|
| 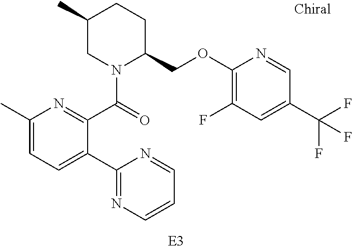 | D11 + 2,3-difluoro- 5- (trifluoromethyl) pyridine | 2-(2-{[(2S,5S)-2-({[3-fluoro-5-(trifluoromethyl)-2- pyridinyl]oxy}methyl)-5-methyl-1- piperidinyl]carbonyl}-6-methyl-3- pyridinyl)pyrimidine UPLC (Acid GEN_QC_SS): rt1 = 0.97 minutes and rt2 = 1.0 min (rotamers present), peaks observed: 490 (M + 1). C₂₄H₂₃F₄N₅O₂ requires 489. ¹H NMR (400 MHz, DMSO-d₆) δ ppm 0.70 (d, 3 H) 0.90-1.02 (m, 3 H) 1.33-1.45 (m, 2 H) 1.53 (br. s., 2 H) 1.62 (br. s., 4 H) 1.70-1.85 (m, 1 H) 1.92-2.04 (m, 1 H) 2.33 (m, 1 H) 2.38 (s, 3 H) 2.56-2.71 (m, 1 H) 3.15 (d, 1 H) 3.90-4.03 (m, 1 H) 4.43 (br. s., 1 H) 4.59 (dd, 1 H) 4.64-4.75 (m, 1 H) 4.81 (m, 2 H) 4.97 (br. s., 1 H) 7.37 (d, 1 H) 7.41-7.55 (m, 3 H) 8.11- 8.30 (m, 3 H) 8.38 (d, 1 H) 8.45-8.57 (m, 2 H) 8.82- 8.96 (m, 4 H) |
| 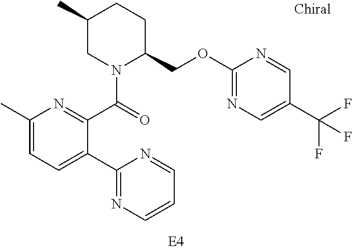 | D11 + 2-chloro-5- (trifluoromethyl) pyrimidine | 2-{[((2S,5S)-5-methyl-1-{[6-methyl-3-(2- pyrimidinyl)-2-pyridinyl]carbonyl}-2- piperidinyl)methyl]oxy}-5- (trifluoromethyl)pyrimidine UPLC (Acid GEN_QC_SS): rt1 = 0.83 minutes and rt2 = 0.87 min (rotamers present), peaks observed: 473 (M + 1). C₂₃H₂₃F₃N₆O₂ requires 472. ¹H NMR (400 MHz, DMSO-d₆) δ ppm 0.70 (d, 3 H) 0.97 (d, 2 H) 1.36 (d, 2 H) 1.62 (br. s., 5 H) 1.77 (br. s., 1 H) 1.98 (d, 1 H) 2.33 (dt, 1 H) 2.38 (s, 2 H) 2.64- 2.70 (m, 1 H) 2.82 (s, 1 H) 3.15 (br. s., 1 H) 3.95 (br. s., 1 H) 4.43 (br. s., 1 H) 4.53-4.70 (m, 2 H) 4.76 (td, 2 H) 4.98 (br. s., 1 H) 7.38 (d, 1 H) 7.42-7.55 (m, 3 H) 8.40 (d, 1 H) 8.49 (d, 1 H) 8.84-8.98 (m, 5 H) 9.13 (d, 2 H) |
| 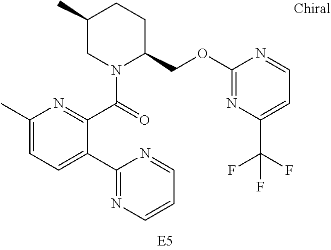 | D11 + 2-chloro-4- (trifluoromethyl) pyridine | 2-{[((2S,5S)-5-methyl-1-{[6-methyl-3-(2- pyrimidinyl)-2-pyridinyl]carbonyl}-2- piperidinyl)methyl]oxy}-4- (trifluoromethyl)pyrimidine UPLC (Acid GEN_QC_SS): rt1 = 0.83 minutes and rt2 = 0.88 min (rotamers present), peaks observed: 473 (M + 1). C₂₃H₂₃F₃N₆O₂ requires 472. ¹H NMR (400 MHz, DMSO-d₆) δ ppm 0.70 (d, 3 H) 0.97 (d, 2 H) 1.38 (br. s., 2 H) 1.52-1.71 (m, 5 H) 1.77 (d, 1 H) 2.00 (d, 1 H) 2.33 (dt, 1 H) 2.38 (s, 2 H) 2.53 (s, 3 H) 2.83 (t, 1 H) 3.15 (d, 1 H) 3.95 (br. s., 1 H) 4.43 (br. s., 1 H) 4.50-4.65 (m, 2 H) 4.70-4.83 (m, 2 H) 4.96 (br. s., 1 H) 7.38 (d, 1 H) 7.43-7.50 (m, 3 H) 7.58 (d, 1 H) 7.70 (d, 1 H) 8.40 (d, 1 H) 8.50 (d, 1 H) 8.81-8.90 (m, 2 H) 8.92-8.98 (m, 2 H) 9.05 (d, 1 H) |
| 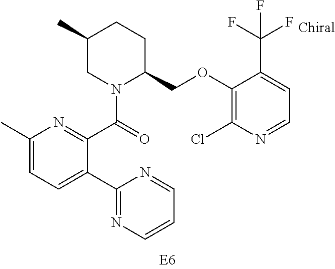 | D11 + 2-chloro-3- fluoro-4- (trifluoromethyl) pyridine | 2-(2-{[(2S,5S)-2-({[2-chloro-4-(trifluoromethyl)-3- pyridinyl]oxy}methyl)-5-methyl-1- piperidinyl]carbonyl}-6-methyl-3- pyridinyl)pyrimidine UPLC (Acid GEN_QC_SS): rt1 = 0.92 minutes and rt2 = 0.94 min (rotamers present), peaks observed: 506 (M + 1). C₂₄H₂₃ClF₃N₅O₂ requires 505. ¹H NMR (400 MHz, DMSO-d₆) δ ppm 0.69 (d, 2 H) 0.92-1.03 (m, 3 H) 1.44 (br. s., 2 H) 1.53-1.83 (m, 5 H) 1.87-1.99 (m, 1 H) 2.10-2.24 (m, 1 H) 2.29-2.46 (m, 5 H) 2.55 (s, 2 H) 2.61-2.79 (m, 2 H) 3.15 (br. s., 1 H) 3.92 (br. s., 1 H) 4.16-4.34 (m, 2 H) 4.34-4.63 (m, 3 H) 5.01 (br. s., 1 H) 7.40-7.52 (m, 3 H) 7.74 (d, 1 H) 7.82 (d, 1 H) 8.39-8.54 (m, 3 H) 8.82 (d, 1 H) 8.90 (d, 2 H) |

### Example 7

### 2-{2-R(2S,5S)-2-{2-[(5-fluoro-2-pyridinyl)oxy]ethyl}-5-methyl-1-piperidinyl)carbonyl]-6-methyl-3-pyridinyl}pyrimidine (E7)

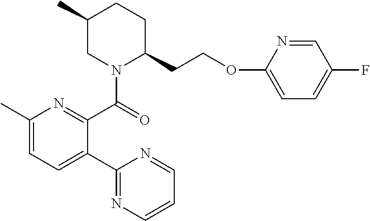

To a suspension of 5-fluoro-2-({2-[(2S,5S)-5-methyl-2-piperidinyl]ethyl}oxy)pyridine D15 (100 mg, 0.420 mmol), DIPEA (0.220 mL, 1.259 mmol) and 6-methyl-3-(2-pyrimidinyl)-2-pyridinecarboxylic acid D9 (391 mg, 0.546 mmol) in DCM (5 ml) stirred under nitrogen at room temperature TBTU (175 mg, 0.546 mmol) was added in one charge. The reaction mixture was stirred at room temperature for 18 hours, then NaHCO₃ aqueous satured solution was added and the aqueous extracted with DCM, the phases were separated on a hydrophobic filter and the combined organic solvent was removed to give the crude product which was purified by flash chromatography (silica 25 g column, gradient elution from Cy to Cy/EtOAc 90/10) the product so obtained was purified by flash chromatography (silica-NH 28 g column, gradient elution from Cy to Cy/EtOAc 35/65) to afford the title compound E7 (65 mg, 0.149 mmol, 35% yield). UPLC (Basic GEN_QC): rt1=0.86 minutes and rt2=0.90 min (rotamers present), peaks observed: 436 (M+1). C₂₄H₂₆FN₅O₂ requires 435. ¹H NMR (500 MHz, CDCl₃) δ ppm 8.70-8.82 (m, 2H), 8.53 (d, 1H), 7.85-7.90 (m, 1H), 7.33 (t, 1H), 7.22-7.28 (m, 1H), 7.15-7.21 (m, 1H), 6.41 (dd, 1H), 4.69 (d, 1H), 4.15-4.30 (m, 2H), 3.75-3.85 (m, 1H), 2.53 (s, 3H), 2.52 (t, 1H), 2.29-2.44 (m, 1H), 1.87-2.01 (m, 1H), 1.12-1.88 (m, 5H), 0.73 (d, 3H).

The following compounds were prepared using a similar procedure to that described for Example 7. Each compound was obtained by amide coupling of 5-fluoro-2-({2-[(2S,5S)-5-methyl-2-piperidinyl]ethyl}oxy)pyridine D15 with the appropriate carboxylic acid. This is provided merely for assistance to the skilled chemist. The starting material may not necessarily have been prepared from the batch referred to.

| No. | Reactants | Characterising data |
|---|---|---|
| 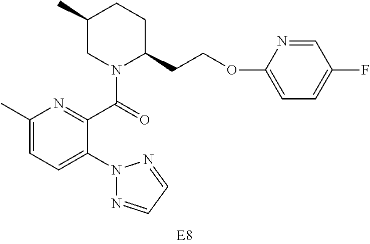 | D15 and D21 | 2-[((2S,5S)-2-{2-[(5-fluoro-2-pyridinyl)oxy]ethyl}-5- methyl-1-piperidinyl)carbonyl]-6-methyl-3-(2H- 1,2,3-triazol-2-yl)pyridine UPLC (Basic GEN_QC): rt1 = 0.89 minutes and rt2 = 0.93 min (rotamers present), peaks observed: 425 (M + 1). C₂₂H₂₅FN₆O₂ requires 424. ¹H NMR (500 MHz, DMSO-d₆) δ ppm 7.99-8.24 (m, 4 H), 7.63- 7.71 (m, 1 H), 7.48-7.53 (m, 1 H), 6.80-6.91 (m, 1 H), 4.77-4.85 (m, 1 H), 4.07-4.40 (m, 2 H), 3.07- 3.14 (m, 1 H), 2.78 (t, 1 H), 2.55 (s, 3 H), 2.17-2.32 (m, 1 H), 1.89-2.03 (m, 1 H), 1.19-1.75 (m, 5 H), 0.92 (d, 3 H). |

### Example 9

### Determination of Antagonist Affinity at Human Orexin-1 and 2 Receptors Using FLIPR

### Cell Culture

Adherent Chinese Hamster Ovary (CHO) cells, stably expressing the recombinant human Orexin-1 or human Orexin-2 receptors or Rat Basophilic Leukaemia Cells (RBL) stably expressing recombinant rat Orexin-1 or rat Orexin-2 receptors were maintained in culture in Alpha Minimum Essential Medium (Gibco/Invitrogen, cat. no.; 22571-020), supplemented with 10% decomplemented foetal bovine serum (Life Technologies, cat. no. 10106-078) and 400 μg/mL Geneticin G418 (Calbiochem, cat. no. 345810). Cells were grown as monolayers under 95%:5% air:CO₂ at 37° C.

The sequences of the human orexin 1, human orexin 2, rat orexin 1 and rat orexin 2 receptors used in this example were as published in Sakurai, T. et al (1998) Cell, 92 pp 573 to 585.

### Measurement of [Ca²⁺]ᵢ Using the FLIPR™

Cells were seeded into black clear-bottom 384-well plates (density of 20,000 cells per well) in culture medium as described above and maintained overnight (95%:5% air:CO₂ at 37° C.). On the day of the experiment, culture medium were discarded and the cells washed three times with standard buffer (NaCl, 145 mM; KCl, 5 mM; HEPES, 20 mM; Glucose, 5.5 mM; MgCl₂, 1 mM; CaCl₂, 2 mM) added with Probenecid 2.5 mM. The plates were then incubated at 37° C. for 60 minutes in the dark with 2 μM FLUO-4AM dye to allow cell uptake of the FLUO-4AM, which is subsequently converted by intracellular esterases to FLUO-4, which is unable to leave the cells. After incubation, cells were washed three times with standard buffer to remove extracellular dye and 30 μL of buffer were left in each well after washing.

Compounds of the invention were tested in a final assay concentration range from 1.66×10⁻⁵ M to 1.58×10⁻¹¹ M. Compounds of the invention were dissolved in dimethylsulfoxide (DMSO) at a stock concentration of 10 mM. These stock solutions were serially diluted with DMSO and 1 μL of each dilution was transferred to a 384 well compound plate. Immediately before introducing compound to the cells, buffer solution (50 μl/well) was added to this plate. To allow agonist stimulation of the cells, a stock plate containing a solution of human orexin A (hOrexin A) was diluted with buffer to final concentration just before use. This final concentration of hOrexin A was equivalent to the calculated EC80 for hOrexinA agonist potency in this test system. This value was obtained by testing hOrexinA in concentration response curve (at least 16 replicates) the same day of the experiment.

The loaded cells were then incubated for 10 min at 37° C. with test compound. The plates were then placed into a FLIPR™ (Molecular Devices, UK) to monitor cell fluorescence (λₑₓ=488 nm, λ_{EM}=540 nm) (Sullivan E, Tucker E M, Dale I L. Measurement of [Ca²⁺]ᵢ using the fluometric imaging plate reader (FLIPR). In: Lambert DG (ed.), Calcium Signaling Protocols. New Jersey: Humana Press, 1999, 125-136). A baseline fluorescence reading was taken over a 5 to 10 second period, and then 10 μL of EC80 hOrexinA solution was added. The fluorescence was then read over a 4-5 minute period.

### Data Analysis

Functional responses using FLIPR were measured as peak fluorescence intensity minus basal fluorescence and expressed as a percentage of a non-inhibited Orexin-A-induced response on the same plate. Iterative curve-fitting and parameter estimations were carried out using a four parameter logistic model and Microsoft Excel (Bowen W P, Jerman J C. Nonlinear regression using spreadsheets. Trends Pharmacol. Sci. 1995; 16: 413-417). Antagonist affinity values (IC₅₀) were converted to functional pKᵢ values using a modified Cheng-Prusoff correction (Cheng Y C, Prusoff W H. Relationship between the inhibition constant (Kᵢ) and the concentration of inhibitor which causes 50 percent inhibition (IC₅₀) of an enzymatic reaction. Biochem. Pharmacol. 1973, 22: 3099-3108).

$$fpKi = - log \frac{( {IC}_{50} )}{{( 2 + {( \frac{[ agonist ]}{( {EC}_{50} )} )}^{n} )}^{1 / n} - 1}$$

Where [agonist] is the agonist concentration, EC₅₀ is the concentration of agonist giving 50% activity derived from the agonist dose response curve and n=slope of the dose response curve. When n=1 the equation collapses to the more familiar Cheng-Prusoff equation.

Compounds of examples 1 to 8 were tested according to the method of example 9. All compounds gave fpKi values from 7.1 to 8.8 at the human cloned orexin-1 receptor and from 7.3 to 8.9 at the human cloned orexin-2 receptor.

1-21. (canceled)

22. A compound of formula (I) 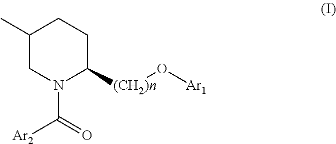
wherein:
Ar₂ is a phenyl, pyridinyl, pyrimidinyl, pyridazinyl or pyrazinyl group, where said group is substituted with a group selected from C₁₋₄alkyl, halo, C₁₋₄alkoxy, haloC₁₋₄alkyl, haloC₁₋₄alkoxy and cyano,
and is additionally substituted with a group Y, where Y is phenyl, pyridinyl, pyrimidinyl, pyridazinyl, pyrazinyl, oxadiazolyl, phenyloxy, pyridinyloxy, pyrimidinyloxy, pyridazinyloxy, pyrazinyloxy, oxadiazolyloxy or a 5 membered heterocyclic group containing 1, 2, 3 or 4 heteroatoms selected from N, O or S, which group Y is optionally substituted with a group selected from C₁₋₄alkyl, haloC₁₋₄alkyl, C₁₋₄alkoxy, haloC₁₋₄alkoxy, cyano and halo;
Ar₁ is a heteroaryl group selected from the group consisting of pyridinyl, pyrimidinyl, pyridazinyl and pyrazinyl, which heteroaryl group is optionally substituted with 1, 2 or 3 substituents independently selected from the group consisting of C₁₋₄alkyl, halo, C₁₋₄alkoxy, haloC₁₋₄alkyl, haloC₁₋₄alkoxy and cyano;
or Ar₁ is an 8 to 10 membered bicyclic heterocyclyl group which bicyclic heterocyclyl group is optionally substituted with C₁₋₄alkyl, haloC₁₋₄alkyl or halo; and
n is 1 or 2;
or a pharmaceutically acceptable salt thereof.

23. The compound, or salt thereof, according to claim 22, where Ar₂ is phenyl, pyridinyl, pyrimidinyl, pyridazinyl or pyrazinyl substituted with a group selected from C₁₋₄alkyl, halo, C₁₋₄alkoxy, haloC₁₋₄alkyl, haloC₁₋₄alkoxy and cyano,
and is additionally substituted with a group Y, where Y is phenyl, pyridinyl, pyrimidinyl, pyridazinyl, pyrazinyl, oxadiazolyl, phenyloxy, pyridinyloxy, pyrimidinyloxy, pyridazinyloxy, pyrazinyloxy, oxadiazolyloxy or a 5 membered heterocyclic group containing 1, 2, 3 or 4 heteroatoms selected from N, O or S, which group Y is optionally substituted with a group selected from C₁₋₄alkyl, haloC₁₋₄alkyl, C₁₋₄alkoxy, haloC₁₋₄alkoxy, cyano and halo;
and Ar₁ is a heteroaryl group selected from the group consisting of pyridinyl, pyrimidinyl, pyridazinyl and pyrazinyl, which heteroaryl group is optionally substituted with 1, 2 or 3 substituents independently selected from the group consisting of: C₁₋₄alkyl, halo, C₁₋₄alkoxy, haloC₁₋₄alkyl, haloC₁₋₄alkoxy and cyano; and
n is 1 or 2.

24. The compound, or salt thereof, according to claim 22, where Ar₂ is pyridinyl substituted with a group selected from C₁₋₄alkyl, halo, C₁₋₄alkoxy, haloC₁₋₄alkyl, haloC₁₋₄alkoxy and cyano, and is additionally substituted with a group Y, where Y is pyrimidinyl which is optionally substituted with a group selected from C₁₋₄alkyl, haloC₁₋₄alkyl, C₁₋₄alkoxy, haloC₁₋₄alkoxy, cyano or halo.

25. The compound, or salt thereof, according to claim 24, where Ar₂ is pyridinyl substituted with C₁₋₄alkyl and is additionally substituted with a group Y, where Y is pyrimidinyl.

26. The compound, or salt thereof, according to claim 22, where Ar₁ is pyridinyl which is substituted with 1 or 2 substituents independently selected from the group consisting of: C₁₋₄alkyl, halo and haloC₁₋₄alkyl.

27. The compound, or salt thereof, according to claim 22, where Ar₂ is pyridinyl substituted with methyl and is additionally substituted with a group Y, where Y is pyrimidinyl; Ar₁ is pyridinyl which is substituted with 1 or 2 substituents independently selected from the group consisting of methyl, fluoro and trifluoromethyl; and n is 1.

28. A compound selected from the group consisting of:
2-(6-methyl-2-{[(2S,5S)-5-methyl-2-({[5-(trifluoromethyl)-2-pyridinyl]oxy}methyl)-1-piperidinyl]carbonyl}-3-pyridinyl)pyrimidine;
2-(6-methyl-2-{[(2S,5S)-5-methyl-2-({[4-(trifluoromethyl)-2-pyridinyl]oxy}methyl)-1-piperidinyl]carbonyl}-3-pyridinyl)pyrimidine;
2-(2-{[(2S,5S)-2-({[3-fluoro-5-(trifluoromethyl)-2-pyridinyl]oxy}methyl)-5-methyl-1-piperidinyl]carbonyl}-6-methyl-3-pyridinyl)pyrimidine;
2-{[((2S,5S)-5-methyl-1-{[6-methyl-3-(2-pyrimidinyl)-2-pyridinyl]carbonyl}-2-piperidinyl)methyl]oxy}-5-(trifluoromethyl)pyrimidine;
2-{[((2S,5S)-5-methyl-1-{[6-methyl-3-(2-pyrimidinyl)-2-pyridinyl]carbonyl}-2-piperidinyl)methyl]oxy}-4-(trifluoromethyl)pyrimidine;
2-(2-{[(2S,5S)-2-({[2-chloro-4-(trifluoromethyl)-3-pyridinyl]oxy}methyl)-5-methyl-1-piperidinyl]carbonyl}-6-methyl-3-pyridinyl)pyrimidine;
2-{2-R(2S,5S)-2-{2-[(5-fluoro-2-pyridinyl)oxy]ethyl}-5-methyl-1-piperidinyl)carbonyl]-6-methyl-3-pyridinyl}pyrimidine; and
2-[((2S,5S)-2-{2-[(5-fluoro-2-pyridinyl)oxy]ethyl}-5-methyl-1-piperidinyl)carbonyl]-6-methyl-3-(2H-1,2,3-triazol-2-yl)pyridine;
or a pharmaceutically acceptable salt thereof.

29. A pharmaceutical composition comprising a) the compound, or salt thereof, according to claim 22, and b) one or more pharmaceutically acceptable carriers.

30. A method of treatment of a disease or disorder where an antagonist of a human orexin receptor is required comprising administering to a subject in need there of an effective amount of the compound, or salt thereof, according to claim 22,
wherein the disease or disorder is a sleep disorder, a depression or mood disorder, an anxiety disorder, a substance-related disorder, or a feeding disorder.

31. The method according to claim 30, wherein the disease or disorder is a sleep disorder.

32. The method according to claim 31, wherein the sleep disorder is selected from the group consisting of Primary Insomnia (307.42), Primary Hypersomnia (307.44), Narcolepsy (347), Breathing-Related Sleep Disorders (780.59), Circadian Rhythm Sleep Disorder (307.45), Dyssomnia Not Otherwise Specified (307.47), Nightmare Disorder (307.47), Sleep Terror Disorder (307.46), Sleepwalking Disorder (307.46), Parasomnia Not Otherwise Specified (307.47), Insomnia Related to Another Mental Disorder (307.42), Hypersomnia Related to Another Mental Disorder (307.44), a Sleep Disorder Due to a General Medical Condition, wherein the sleep disorder is a sleep disturbance associated with a medical condition selected from the group consisting of a neurological disorder, neuropathic pain, restless leg syndrome, heart disease and lung disease, Insomnia Type Substance-Induced Sleep Disorder, Hypersomnia Type Substance-Induced Sleep Disorder, Parasomnia Type Substance-Induced Sleep Disorder, Mixed Type Substance-Induced Sleep Disorder, Sleep Apnea and Jet-Lag Syndrome.
